(19) United States
(12) Patent Application Publication
(10) Pub. No.: US 2026/0041758 A1
(43) Pub. Date: Feb. 12, 2026

(54) IMMUNIZATION STRATEGIES TO MORE NATURALLY GUIDE THE MATURATION OF ANTIBODIES AGAINST HUMAN IMMMUNODEFICIENCY VIRUS (HIV) IN HIV-INFECTED SUBJECTS

(71) Applicant: Fred Hutchinson Cancer Center, Seattle, WA (US)

(72) Inventors: Leonidas Stamatatos, Seattle, WA (US); Lawrence Corey, Mercer Island, WA (US)

(73) Assignee: Fred Hutchinson Cancer Center, Seattle, WA (US)

(21) Appl. No.: 19/102,240

(22) PCT Filed: Aug. 9, 2023
(86) PCT No.: PCT/US2023/071940

§ 371 Date: Feb. 7, 2025

Related U.S. Application Data
(60) Provisional application No. 63/370,910, filed on Aug. 9, 2022.

(51) Int. Cl. A61K 39/21; A61K 39/00; C07K 14/005

(52) U.S. Cl. CPC A61K 39/21; C07K 14/005; A61K 2039/545; A61K 2039/55505; A61K 2039/645; C12N 2740/16122; C12N 2740/16134

(57) ABSTRACT

Immunization strategies to naturally guide the maturation of antibodies against the human immunodeficiency virus (HIV) in HIV-infected subjects are described. The strategies include immunization utilizing an HIV envelope protein (Env) that binds germline (gl) B cells combined with taking HIV-infected subjects off of anti-viral medications, allowing natural virus to guide the maturation of gl B cells activated by the immunization. When B cells effectively mature against the HIV virus. HIV-infected subjects may remain off of anti-viral medications.

### CROSS-REFERENCE TO RELATED APPLICATIONS

This application is a U.S. National Phase Patent Application based on International Patent Application No. PCT/US2023/071940, filed on Aug. 9, 2023, which claims priority to U.S. Provisional Patent Application No. 63/370,910 filed Aug. 9, 2022, each of which is incorporated herein by reference in its entirety as if fully set forth herein.

### REFERENCE TO SEQUENCE LISTING

The Sequence Listing associated with this application is provided in XML format in lieu of a paper copy and is hereby incorporated by reference into the specification. The name of the file containing the Sequence Listing is 3DA5777.XML. The file is 151,981 bytes, was created on Jan. 23, 2025, and is being submitted electronically via Patent Center.

### FIELD OF THE DISCLOSURE

The current disclosure provides immunization strategies to more naturally guide the maturation of antibodies against the human immunodeficiency virus (HIV) in HIV-infected subjects. The strategies include immunization utilizing an HIV envelope protein (Env) that binds germline (gl) B cells as in combination with taking HIV-infected subjects off of anti-viral medications, allowing natural virus to guide the maturation of immunization activated gl B cells. When B cells effectively mature against the HIV virus, HIV-infected subjects may remain off of anti-viral medications.

### BACKGROUND OF THE DISCLOSURE

Acquired Immunodeficiency Syndrome (AIDS) is characterized by immunosuppression that results in opportunistic infections and malignancies, wasting syndromes, and central nervous system degeneration. Destruction of CD4+ T-cells, which are critical to immune defense, is a major cause of the progressive immune dysfunction that is the hallmark of AIDS disease progression. The loss of CD4+ T cells seriously impairs the body's ability to fight most pathogens, but it has a particularly severe impact on the defenses against viruses, fungi, parasites and certain bacteria, including mycobacteria.

AIDS is caused by infection with the human immunodeficiency virus (HIV). HIV infection begins when a portion of the virus' envelope protein (Env), gp120, binds to CD4 and other receptors on the surface of an infected subject's CD4+ T-cells and other immune system cells. The bound virus then fuses with the bound infected subject's cell and reverse transcribes its RNA genome. The resulting viral DNA integrates into the infected subject's cell's genome and begins to produce new viral RNA, resulting in new viral proteins and virions. The virions leave the originally infected cell to infect new cells. This process kills the originally infected cell.

Antibodies are proteins that can provide protection against pathogens, such as viruses. In some instances, antibodies are protective because they bind to an invading pathogen and interfere with its normal function. For example, many protective antibodies bind to a portion of a virus that blocks its ability to bind to and/or enter an infected subject's cells.

Vaccines are designed to increase the immunity of a subject against a particular pathogen by exposing subjects to an innocuous form or portion of the pathogen that will not lead to an active and/or on-going infection. Exposure to the form or portion of the pathogen stimulates B cells to produce antibodies against the targeted pathogen.

Each B cell expresses a unique antibody with unique specificity for a particular epitope on a protein. The unique antibody expressed by each B cell is generated randomly through genetic recombination. A germline (gl) B cell refers to an immature B cell before it has come into contact with its epitope. gl B cells express membrane-bound antibodies (also called B cell receptors or BCR). When a BCR encounters and binds its particular epitope, the B cell begins to rapidly proliferate and mature. During proliferation and maturation, the B cell's antibody genes undergo somatic hypermutation, which serves to increase the affinity of the B cell's antibody to its initial epitope. The increase in affinity of epitope binding that occurs during B cell maturation is required for effective protection against the pathogen. A single gl B cell is able to undergo dozens of cell divisions to create thousands of antibody-secreting B cells and memory B cells expressing the same antibody, or a related antibody that has been mutated to improve binding to the pathogen.

For decades, researchers have been trying to develop a vaccine that can induce B cells to produce antibodies that are effective to protect against HIV. But all efforts to induce protective antibodies to date have not led to sufficiently effective and on-going protection.

VRC01-class antibodies are among the most broad and potent neutralizing antibodies known against HIV. Balazs et al., Nature. 2012; 481(7379):81-4; Balazs et al., Nature Medicine. 2014; 20(3):296-300; Shingai et al., Journal of Experimental Medicine. 2014; 211(10):2061-74. VRC01-class antibodies are important targets for an HIV-1 vaccine because they are among the most broad and potent neutralizing antibodies known; their passive administration to non-human primates prior to SHIV-exposure or their active expression in humanized mice prior to HIV-1 exposure leads to protection from infection (in Balazs et al., Nature 481, 81-84, (2012). Balazs et al., (Nat Med 20, 296-300, (2014)), Shingai et al., J Exp Med 211, 2061-2074, (2014), and two phase 3 clinical trials (HVTN 703/704) demonstrated that a member of that antibody class (mAb VRC01) prevents HIV-1 acquisition from VRC01-sensitive tier 2 viruses (in Corey et al., N Engl J Med 384, 1003-1014, (2021)). The development of an immunization protocol that would reproducibly elicit VRC01-like bNAbs will be of high clinical significance to the development of such a vaccine.

Engineered HIV Env proteins that bind to glBCRs (including glVRC01 BCRs) and activate B cells have been developed. McGuire et al., J Exp Med 210(4), 655-663 (2013) and WO2016/154422 describe an engineered Env protein (the ‘426c core’) that binds and activates the inferred gl forms of BCR, including diverse VRC01-class bNAbs. This protein was derived from the Clade C virus 426, which was isolated from an HIV patient 90 days post-infection. Jardine et al., Science, 340 (6133), 711-716 (2013) and WO2016/205704 describe “engineered outer domain” or “eOD” Env proteins that bind glVRC01 BCR. Medina-Ramierz et al., J Exp Med 214(9), 2573-2590 (2017) describes the engineered BG505 SOSIP.664 glycoprotein, BG505 SOSIP.v4.1-GT1 trimer. While immunization with these engineered HIV Env proteins alone stimulate glBCR that can lead to broadly-neutralizing antibodies against HIV, the stimulated glBCR did not sufficiently mature to create broadly-neutralizing antibodies.

One of the important reasons for the lack of complete success to date is due to the fact that the appropriate maturation of glBCRs (including VRC01 BCRs) requires that somatic hypermutations during maturation effectively circumvent steric binding constraints on HIV's Env protein. For example, HIV-1 has evolved to avoid detection by glVRC01 B cells. HIV-1 avoids detection by glVRC01 B cells through the presence of specific N-linked glycosylation sites (NLGS) within the gp120 protein. One such NLGS is found at position 276 of the Env. As a consequence, recombinant Env proteins derived from diverse HIV-1 isolates are ineffective in binding to and stimulating B cells that express the glBCR forms of VRC01-class bNAbs. Targeted disruption of certain conserved NLGS, however, permits binding and activation gl B cell lines expressing BCRs of two clonally-related VRC01-class bNAbs, VRC01 and NIH45-46. These two BCRs represent a small subset of potential VRC01-class antibody progenitors. Thus, designing immunogens capable of recognizing a larger group of glBCR, including glVRC01-class BCRs should increase the chances of activating rare, naïve gl B cells during human immunization.

An even newer approach in the on-going attempt to elicit bNAbs against HIV through vaccination is based on the ‘germline-targeting’ approach described in the preceding paragraph combined with subsequently guiding the maturation of the first wave of germline antibodies towards their broad neutralizing form along specific evolutionary pathways, using specifically designed ‘booster’ engineered Env. Several attempts have been made to guide the maturation of glBCR stimulated with engineered Env. These strategies include, for example, Tian et al., (2016 Cell, 166, 1471-1484) who administered:
(i) multimerized eOD, followed by
(ii) multimerized 426c core, followed by
(iii) 426c core monomer glycosylated at N463, followed by
(iv) 426c core monomer glycosylated at N463 and N460, followed by
(v) 426c core monomer glycosylated at N463, N460, and N276 followed by
(vi) a soluble wild-type trimeric 426c (i.e., a natural HIV Env); and
Briney et al., (2016 Cell 166, 1459-1470) who administered either:
(A-i) multimerized eOD, followed by
(A-ii) multimerized BG505 GTC core (this protein is similar to the 426c core, but generated from a different strain of HIV) followed by
(A-iii) a soluble wild-type trimeric BG505, but for removal of the NLGS site at N276, followed by
(A-iv) a repeat administration of the soluble wild-type trimeric BG505, but for removal of the NLGS site at N276
or
(B-i) multimerized eOD, followed by
(B-ii) a soluble wild-type trimeric BG505, but for removal of NLGS sites N460, N463, and N276 followed by
(B-iii) a soluble wild-type trimeric BG505, but for removal of the NLGS site at N276, followed by
(B-iv) a repeat administration of the soluble wild-type trimeric BG505, but for removal of the NLGS site at N276.

Unfortunately, none of these approaches elicited the maturation of antibodies capable of broadly neutralizing HIV. Tian et al., Cell. 166(6): 1471-84 e18 (2016); Briney et al., Cell 166, 1459-1470, (2016); Chen et al., Immunity, 54(2), 324-329 (2021).

Many immunization efforts to date have focused on non-HIV infected subjects to prevent infection with the virus. This may be in part due to the fact that many successful anti-retroviral therapies (ART) have greatly improved the outcome for HIV-infected subjects. For subjects receiving ART, the HIV virus may remain latent for several decades or more, and most patients on ART do not have detectable HIV in their blood. However, ART does not cure HIV infection, and once therapy is stopped, HIV in blood rebounds to its pre-treatment level. The ability to stop treatment without viral rebound would be beneficial because long-term treatment with ART is associated with other serious health considerations such as bone or renal toxicity, insulin resistance, and accelerated cardiovascular disease. However, stopping treatment is discouraged due to the likelihood of viral rebound from the latent HIV reservoir.

### SUMMARY OF THE DISCLOSURE

The current disclosure provides immunization strategies to successfully guide the maturation of antibodies against human immunodeficiency virus (HIV) in HIV-infected subjects. The immunization strategies have two key components: (i) administration of an engineered HIV envelope protein (Env) capable of stimulating germline (gl) B cells (including glVRC01 B cells that target the CD4-binding site) that can mature to produce broadly neutralizing antibodies against HIV to an HIV-infected subject; and (ii) taking the HIV-infected subject off of anti-viral medications, so that natural virus can temporarily rebound to guide the maturation of the stimulated gl B cells. When B cells effectively mature against the HIV virus, HIV-infected subjects may remain off or be taken off of anti-viral medications helping to alleviate many of the negative side effects associated with anti-retroviral therapy (ART). A similar strategy can be employed with immunogens that aim at eliciting broadly neutralizing antibodies (bNAbs) of other specificities, such as against the N332 site, the gp41 membrane proximal external region (MPER), the V2 apex, and other sites or regions. This approach may lead to the identification of the precise viral Envs that guide the maturation of bNAbs during infection and thus lead to the development of the corresponding immunogens to be used in uninfected subjects.

### DETAILED DESCRIPTION

Acquired Immunodeficiency Syndrome (AIDS) is characterized by immunosuppression that results in opportunistic infections and malignancies, wasting syndromes, and central nervous system degeneration. Destruction of CD4+ T-cells, which are critical to immune defense, is a major cause of the progressive immune dysfunction that is the hallmark of AIDS disease progression. The loss of CD4+ T cells seriously impairs the body's ability to fight most pathogens, but it has a particularly severe impact on the defenses against viruses, fungi, parasites and certain bacteria, including mycobacteria.

AIDS is caused by infection with human immunodeficiency virus (HIV). The HIV genome encodes several structural proteins. The env gene encodes the viral envelope glycoprotein (Env), a 160-kilodalton (kDa) protein. Env is cleaved into an external 120-kDa envelope glycoprotein (gp120) and a transmembrane 41-kDa envelope glycoprotein (gp41). gp120 and gp41 are required for HIV to infect cells.

Mature gp120 wildtype (wt) proteins have 500 amino acids in the primary sequence. gp120 is heavily N-glycosylated giving rise to an apparent molecular weight of 120 kDa. The protein includes five conserved regions (C1-C5) and five regions of high variability (V1-V5). Exemplary sequences of wt gp 120 proteins are found in GenBank® (United States Department of Health and Human Services, Bethesda, MD), for example accession numbers AAB05604 and AAD12142. It is understood that there are numerous variations in the sequence of gp120 from what is given in these examples. In particular embodiments, and based on the Hxb2 sequence, V5 includes residues 458-466. One of ordinary skill in the art recognizes, however, that V5 varies among different strains in its precise length, amino acid composition, and glycosylation sites. Reference to residues and mutation positions herein refer to Hxb2 numbering, unless clearly noted to the contrary.

HIV infection begins when gp120, binds to CD4 and other receptors on the surface of a host's target immune system cells (e.g., CD4+ T-cells, macrophages and dendritic cells). The bound virus then fuses with the target cell and reverse transcribes its RNA genome. The resulting viral DNA integrates into the host cell's genome and begins to produce new viral RNA, resulting in new viral proteins and virions. The virions leave the originally infected cell to then infect new cells. This process kills the originally infected cell.

Antibodies are proteins that can provide protection against pathogens. Antibodies can bind to a pathogen and are protective when this binding interferes with the normal function of a pathogen. For example, many protective antibodies bind to a portion of a pathogen that blocks the pathogen from entering cells. Antibodies can be attached to the surface of B cells (known as B cell receptors or BCR) but exert most of their protective functions when secreted into the blood.

HIV-1 neutralizing antibodies are antibodies capable of neutralizing HIV's infection of host cells. Kumar et al., (ACS Omega 2023, 8, 8, 7252-7261) provides an overview of human HIV-1 neutralizing antibodies against diverse epitopes of HIV-1. HIV antibodies generally target four major areas of the Env protein: (i) the portion of gp41 that is external to the cell, but proximal to the cell membrane; (ii) the CD4 receptor-binding site (CD4-BS) of gp120; (iii) two sites including both carbohydrate and amino acid moieties, one at the base of the “V3” loop and another on the “V1/V2” loops of the gp120 subunit; and (iv) regions spanning elements of both gp 120 and gp41.

Based on their ontogenies and mode of recognition, CD4-BS bNAbs are grouped into two major types: (i) heavy chain complementary determining region three (CDRH3)-dominated; and (ii) variable heavy (VH)-gene-restricted. Antibodies that make contact primarily through their CDRH3 regions are further subdivided into the CH103, HJ16, VRC13 and VRC16 classes while the VH-gene-restricted Abs include the VRC01-and the 8ANC131-class antibodies.

Vaccines are designed to increase the immunity of a subject against a particular infection by stimulating B cells to produce antibodies against the targeted infectious agent. Each B cell expresses a unique antibody with unique epitope specificity. The unique antibody expressed by each B cell is generated randomly through genetic recombination. A germline (gl) B cell refers to a B cell that has not yet come in contact with its epitope. gl B cells express a membrane-bound BCR. When the BCR binds its particular epitope, the B cell can rapidly proliferate and mature. During proliferation and maturation, the antibody genes undergo somatic hypermutation, which serves to increase the affinity of epitope binding. The increase in affinity of epitope binding that occurs during B cell maturation is required for effective protection against the pathogen. A single naïve B cell is able to undergo dozens of cell divisions to create thousands of antibody-secreting B cells and memory B cells expressing the same antibody, or a related antibody that has been mutated to improve binding to the pathogen. This binding can lead to activation of the B cell and production of protective antibodies.

For decades, researchers have been trying to develop a vaccine that can induce B cells to produce antibodies that are effective to protect against HIV. But all efforts to induce protective antibodies to date have not led to sufficiently effective and on-going protection.

One of the many important reasons for the lack of success is thought to be the inability of the Env proteins used as immunogens to engage gl B cell BCRs that encode, for example, the gl of VRC01-class antibodies (e.g., “immature” or not fully developed Abs). Indeed, maturation of these antibodies to full neutralizing Abs requires that they circumvent steric constraints on Env through extensive somatic hypermutation. For example, HIV-1 has evolved to avoid detection by gl B cells that give rise to VRC01-class bNAbs through development of specific N-linked glycosylation sites (NLGS) (for example, in Loop D and V5 of the gp120 subunit). As a consequence, recombinant Env proteins derived from diverse HIV-1 isolates are ineffective in binding to and stimulating B cells engineered to express the glBCR forms of VRC01-class bNAbs in vitro. Targeted disruption of conserved NLGS at position 276 in Loop D, and at positions 460 and 463 in V5 of the 426c clade C Env, however, permits binding and activation gl B cell lines expressing BCRs of two clonally-related VRC01-class bNAbs, VRC01 and NIH45-46 in vitro. These two BCRs represent a small subset of potential VRC01-class antibody progenitors. Thus, designing immunogens capable of recognizing a larger group of glVRC01-class BCRs should increase the chances of activating rare, naïve glVRC01-class B cells during human immunization. In particular embodiments, loop D includes residues 275-283.

During the past decades, the generation of novel reagents for the isolation of individual B cells and the establishment of high-throughput methodologies led to the characterization of a plethora of new bNAbs from HIV-1-infected subjects. The structural characterization of such antibodies, combined with information of their ontogenies, have vastly improved the understanding on how such antibodies are generated during natural infection and how they interact with Env. For example, when the VH and VL domains of certain bNAbs (b12, 2G12 and 2F5) are reverted to their predicted, inferred germline forms (from here onward termed ‘germline’ for brevity, unless otherwise noted), those antibodies (Abs) no longer bound the dual-tropic 89.4 Env (Xiao et al., Biochemical and Biophysical Research Communications. 2009; 390(3):404-9). This was true for many of the bNAbs that have been isolated since, irrespective of their epitope-specificity and their VH/VL-derivation (Jardine et al., Science. 2013; 340(6133):711-6; McGuire et al., Journal of Experimental Medicine. 2013 ;210(4):655-63; Hoot et al., PLoS Pathogens. 2013; 9(1):e1003106; Klein et al., Cell. 2013; 153(1):126-38; Diskin et al., Science. 2011; 334(6060):1289-93).

A hypothesis was put forth that commonly available recombinant Envs are ineffective in eliciting bNAbs because they do not initiate the very first step of that process (Jardine et al., Science. 2013; 340(6133):711-6; McGuire et al., Journal of Experimental Medicine. 2013; 210(4):655-63). It was also hypothesized that during natural HIV-1 infection, rare viral clones that express Envs with particular features emerge and that they initiate the activation of naïve B cells that express germline BCRs that eventually produce bNAbs. This was confirmed through studies showing that Env clones with particular features can emerge during infection and engage the germline BCRs of bNAbs and activate the corresponding naïve B cells (Liao et al., Nature. 2013; 496(7446):469-76; Doria-Rose et al., Nature. 2014; 509(7498):55-62). The subsequent maturation of these germline antibodies into their broad neutralizing forms requires the emergence of viruses expressing Env variants of the original ‘germline-binding’ one. In cases where the ‘natural’ Envs that are linked with the development of a particular type of bNAb are known, an immunization scheme can be developed based on those ‘natural’ Envs. In many cases however, such natural Envs are not known. Such is the case of the VRC01-class bNAbs. In those cases, de novo Env immunogens must be designed.

The 426c core activates B cells expressing the germline BCRs of two VRC01-class antibodies, 3BNC60 and 12A21 (Dosenovic et al., Cell. 2015; 161(7):1505-15; McGuire et al., Nature communications. 2016; 7:10618), among other glBCR that produce CD4-BS antibodies.

The 426c core includes modifications to the clade C 426c Env to allow binding the gl forms of BCR, including VRC01-class antibodies (McGuire et al., Nature communications. 2016; 7:10618). In particular embodiments, the 426c core is based on the gp 120 subunit of the 426c Env; it lacks the variable regions 1, 2 and 3 and lacks three key, conserved NLGS (position N276 in Loop D and positions N460 and N463 in V5). Thus, the 426c core includes elements of both the inner and outer domains of gp120. In that, it differs from the engineered outer domain of gp120 (eOD) construct, which only expresses elements of the outer gp120 domain (Jardine et al., Science. 2013; 340(6133):711-6). The structures of 426c core bound to germline 3BNC60 and germline NIH45-46 are reported in Scharf et al., (eLife. 2016; 5. doi: 10.7554/eLife. 13783) and a high-resolution structure (2.4 Å) of the germline VRC01 antibody bound to the 426c core was recently obtained.

Several studies have shown that the instillation of bNAbs in HIV infected persons can suppress viral replication usually transiently; in most people breakthrough with resistant strains may occur; in others, magnitude of the effect or as yet unexplained reasons account for this. This approach is expensive and requires frequent infusions. A more practical approach, clinically, is to induce the development of these antibodies in HIV infected persons so that their production results in functional containment of the virus, obviating the need for continuous antiviral therapy, especially among persons with poor compliance to current therapy. Many immunization efforts to date have focused on non-HIV infected subjects to prevent infection with the virus. This may be in part due to the fact that many successful anti-retroviral therapies (ART) have greatly improved the outcome for HIV-infected subjects. For subjects receiving ART, the HIV virus may remain latent for several decades or more, and most patients on ART do not have detectable HIV in their blood. However, ART does not cure HIV infection, and once therapy is stopped, HIV in blood rebounds to its pre-treatment level. The ability to stop treatment without viral rebound would be beneficial because long-term treatment with ART is associated with other serious health considerations such as bone or renal toxicity, insulin resistance, and accelerated cardiovascular disease. However, stopping treatment is discouraged due to the likelihood of viral rebound from the latent HIV reservoir.

The current disclosure provides immunization strategies to successfully guide the maturation of antibodies against human immunodeficiency virus (HIV) in HIV-infected subjects. The immunization strategies have two key components: (i) administration of an engineered HIV envelope protein (Env) capable of stimulating germline (gl) B cells (including glVRC01 B cells) that can mature to produce broadly neutralizing antibodies against HIV to an HIV-infected subject taking (or who has recently taken) anti-viral medications to treat HIV; and (ii) taking the HIV-infected subject off of the anti-viral medications, so that natural virus can temporarily rebound to guide the maturation of the stimulated gl B cells. When B cells effectively mature against the HIV virus, HIV-infected subjects will develop bNAbs that are capable of containing both replicating viruses and viruses in latent/persistent reservoirs. This strategy allows subjects to remain off of the anti-viral medications (or be re-taken off if treatment was re-initiated in the interim), thereby helping alleviate many negative side effects associated with ART.

Aspects of the disclosure are now described in more detail as follows: (i) Antibodies and Epitopes; (ii) VRC01 Antibodies; (ii-a) The Heavy Chain (HC) of VRC01-Class Antibodies; (ii-b) The Light Chain (LC) of VRC01-Class Antibodies; (iii) Vaccination Protocols in HIV-Infected Subjects; (iv) Multimerization of Env; (v) Vaccine Adjuvants; (vi) Compositions; (vii) Kits; (viii) Methods of Use; (ix) Exemplary Embodiments; and (x) Closing Paragraphs. These headings are provided for organizational purposes only and do not limit the scope or interpretation of the disclosure.

(i) Antibodies and Epitopes. Naturally occurring antibody structural units include a tetramer. Each tetramer includes two pairs of polypeptide chains, each pair having one light chain and one heavy chain. The amino-terminal portion of each chain includes a variable region that is responsible for antigen recognition and epitope binding. The variable regions exhibit the same general structure of relatively conserved framework regions (FR) joined by three hyper variable regions, also called complementarity determining regions (CDRs). The CDRs from the two chains of each pair are aligned by the framework regions, which enables binding to a specific epitope. From N-terminal to C-terminal, both light and heavy chain variable regions include the domains FR1, CDR1, FR2, CDR2, FR3, CDR3 and FR4. The assignment of amino acids to each domain is typically in accordance with the definitions of Kabat Sequences of Proteins of Immunological Interest (National Institutes of Health, Bethesda, Md. (1987 and 1991)), or Chothia & Lesk, J. Mol. Biol., 196:901-917 (1987); Chothia et al., Nature, 342:878-883 (1989). Kabat numbering is used herein unless specifically noted otherwise.

The carboxy-terminal portion of each chain defines a constant region that can be responsible for effector function. Examples of effector functions include: C1q binding and complement dependent cytotoxicity (CDC), antibody-dependent cell-mediated cytotoxicity (ADCC), antibody-dependent phagocytosis (ADCP), down regulation of cell surface receptors (e.g., B cell receptors), and B cell activation.

Within full-length light and heavy chains, the variable and constant regions are joined by a “J” region of amino acids, with the heavy chain also including a “D” region of amino acids. See, e.g., Fundamental Immunology, Ch. 7 (Paul, W., ed., 2nd ed. Raven Press, N.Y. (1989).

Human light chains are classified as kappa (κ) and lambda (λ) light chains. Heavy chains are classified as mu, delta, gamma, alpha, or epsilon, and define the antibody's isotype as IgM, IgD, IgG, IgA, and IgE, respectively. IgG has several subclasses, including, IgG1, IgG2, IgG3, and IgG4. IgM has subclasses including IgM1 and IgM2. IgA is similarly subdivided into subclasses including IgA1 and IgA2.

Antibodies bind epitopes on antigens. An antigen refers to a molecule or a portion of a molecule capable of being bound by an antibody. An epitope is a region of an antigen that is bound by the variable region of an antibody. An epitope includes specific amino acids that contact the variable region of an antibody. Epitope determinants can include chemically active surface groupings of molecules such as amino acids, sugar side chains, phosphoryl or sulfonyl groups, and can have specific three-dimensional structural characteristics, and/or specific charge characteristics.

An “epitope” includes any determinant capable of being bound by an antibody. An epitope is a region of a molecule that is bound by an antibody that targets that region of the molecule, and when that region of the molecule is a protein, includes specific residues that directly contact the binding protein. In particular embodiments, an “epitope” denotes the binding site on a protein target bound by a corresponding antibody. The antibody either binds to a linear epitope, (e.g., an epitope including a stretch of 5 to 12 consecutive amino acids), or the antibody binds to a three-dimensional structure formed by the spatial arrangement of several short stretches of the protein target. Three-dimensional epitopes recognized by an antibody, e.g., by the epitope recognition site or paratope of an antibody or antibody fragment, can be thought of as three-dimensional surface features of an epitope molecule. These features fit precisely (in)to the corresponding binding site of the antibody and thereby binding between the antibody and its target protein is facilitated. In particular embodiments, an epitope can be considered to have two levels: (i) the “covered patch” which can be thought of as the shadow an antibody would cast; and (ii) the individual participating side chains and backbone residues. Binding is then due to the aggregate of ionic interactions, hydrogen bonds, and hydrophobic interactions.

(ii) VRC01-Class Antibodies. As indicated previously, based on their ontogenies and mode of recognition, CD4-BS bNAbs are grouped into two major types: CDRH3-dominated (the most common way antibodies bind their epitopes) and VH-gene-restricted (Zhou et al., Cell. 2015; 161(6):1280-92). Antibodies that make contact primarily through their CDRH3 regions are further subdivided into the CH103, HJ16, VRC13 and VRC16 classes, while the VH-gene-restricted antibodies, which make contact primarily through their CDRH2 domains, include the VRC01- and the 8ANC131-classes (derived from VH1-2 and VH1-46, respectively). At least 29 VRC01-class antibodies have been isolated from at least nine HIV-1+ subjects (Diskin et al., Science. 334(6060):1289-93 (2011); Wu et al., Science. 333(6049):1593-602 (2011); Zhou et al., Immunity. 39 (2): 245-58 (2013); Huang et al., Immunity. 45(5): 1108-21 (2016); Zhou et al., Cell. 161(6):1280-92 (2015); Kwong & Mascola, Immunity. 37(3):412-25 (2012); Sajadi et al., Cell. 173(7):1783-1795 (2018); Umotoy et al., Immunity. 51(1):141-154 (2019); Barnes et al., Sci Adv 8, eabp8155, (2022

(ii-a) The Heavy Chain (HC) of VRC01-Class Antibodies. All known VRC01-class antibodies are derived from one of the five VH1-2 alleles, the VH1-2*02 allele. Three amino acids, Trp50 heavy chain (HC), Asn58HC and Arg71HC, present in the CDRH2 domain of VRC01-class antibodies (i.e., they are encoded by the germline VH1-2 gene segment) make key contacts with Env (Scharf et al., Proceedings of the National Academy of Sciences of the United States of America. 2013; 110(15):6049-54; West et al., Proceedings of the National Academy of Sciences of the United States of America. 2012; 109(30):E2083-90; Zhou et al., Immunity. 2013; 39(2):245-58). Structural information has revealed the reasons why these three amino acids are critically important for the interaction of the *02 allele with Env: Trp50_{HC} makes contact with the conserved amino acid in Loop D, Asn280; Asn58_{HC} makes contact with the conserved amino acid Arg456 in V5; and Arg71_{HC} makes a key contact with amino acid Asp368 in the CD4-BS. Despite the extensive amino acid changes that occur during affinity maturation of these Abs, these three key HC amino acids remain unaltered (Scharf et al., Proceedings of the National Academy of Sciences of the United States of America. 2013; 110(15):6049-54; Zhou et al., Cell. 2015; 161(6):1280-92; Scharf et al., eLife. 2016; 5. doi: 10.7554/eLife. 13783). In addition to allele *02, alleles *03 and *04 also express these three amino acids.

It is also noteworthy that mature VRC01-class Abs have an 11-18 amino acid long CDRH3 and most have a Trp that is located 5 amino acids before the start of FW4 (Trp 100BHC on VRC01 numbering). This Trp interacts with Asn279 gp120 via hydrogen-bonding (Scharf et al., Proceedings of the National Academy of Sciences of the United States of America. 2013; 110(15):6049-54). This Trp is present in the germline CDRH3 of VRC01-class Abs and are expressed on naïve B cells in HIV-1-subjects (Yacoob et al., Cell Reports. 2016; 17(6):1560-70).

(ii-b) The Light Chain (LC) of VRC01-Class Abs. Only a few LC families (K3-20, K3-15, K1-33, K1-5, and λ2-14) are presently known to pair with VH1-2*02 to generate VRC01-class antibodies. Importantly, all the LCs associated with VRC01-class bNAbs express an unusually short (5 amino acid long) CDRL3 region (Zhou et al., Immunity. 2013; 39(2):245-58; Zhou et al., Cell. 2015; 161(6):1280-92; Kwong & Mascola, Immunity. 2012; 37(3):412-25). Less than 0.05% of LCs with these properties are present in the human naïve B cell repertoire (Jardine et al., Science. 2016; 351(6280):1458-63; Sok et al., Science. 2016; 353(6307):1557-60). The particular angle of approach of VRC01-class Abs requires such a short CDRL3; otherwise, these Abs will not bind Env because of steric clashes with Loop D and V5. Thus, without being bound by theory, the short CDRL3 is presently believed not to be the result of somatic hypermutation but has to exist in the germline form of these antibodies (Zhou et al., Immunity. 2013; 39(2):245-58). Accordingly, one of the main goals of ‘germline-targeting’ immunogens is to select for B cells expressing VH1-2*02 VH paired with LCs with 5 amino acid long CDRL3s. Within the 5 amino acid stretch, a key feature of the mature VRC01 Abs is the presence of a negatively charged amino acid, glutamic acid, at position 96. Glu96_{LC} makes key contacts with the V5 loop and Loop D and is one of the amino acids that are linked with the neutralizing activities of VRC01-class Abs. So, ideally, a targeting immunogen should select for LCs with a 5 amino acid long CDRL3 that includes a Glu96. The CDRL1 domains of the mature VRC01-class Abs are also involved in the interaction of these Abs with Env. The mature CDRL1 domains are either shorter (by 2-6 AA) than the corresponding germline domains or contain multiple glycines which provide chain flexibility (Scharf et al., Proceedings of the National Academy of Sciences of the United States of America. 2013; 110(15):6049-54; Zhou et al., Immunity. 2013; 39(2):245-58; Scharf et al., eLife. 2016; 5. doi: 10.7554/eLife.13783). The combination of an unusually short CDRL3 (present at the germline level) with a shortening of the CDRL1 (acquired during affinity maturation) allows the mature VRC01-class antibodies to bypass several key steric clashes with Env, in particular carbohydrate moieties that are located in Loop D (conserved position N276). New information from the structural analysis of several germline VRC01-class antibodies bound to Env-derived proteins, suggests that the CDRL1 amino acid shortening may not be important for the recognition of Env by glVRC01-class antibodies. In sum, VRC01-class germline antibodies exhibit preformed antigen-binding and conformations and affinity maturation that result in increased induced-fit recognition (Scharf et al., eLife. 2016; 5. doi: 10.7554/eLife. 13783).

(iii) Vaccination Protocols in HIV-Infected Subjects. According to certain aspects of the current disclosure, an administered immunogen (e.g., an HIV Env) must be capable of binding and activating glVRC01 B cells.

In particular embodiments, an HIV Env includes the 426c core. In particular embodiments, the 426c core includes an HIV Env protein with the following mutations, modifications, and characteristics: mutations N460D, N463D, S278R, G471S, V65C, and S115C; no mutation at position 276; removal of V1 and V2; V3 replacement with a flexible linker; an N-terminal truncation before 44; and a C-terminal truncation after 494. In particular embodiments, the 426c core includes the sequence:

(SEQ ID NO: 1)
VWKEAKTTLFCASDAKAYEKECHNVWATHACVPTDPNPQEVVLENVTEN
FNMWKNDMVDQMQEDVISIWDQCLKPCVKLTNTSTLTQACPKVTFDPIP
IHYCAPAGYAILKCNNKTFNGKGPCNNVSTVQCTHGIKPVVSTQLLLNG
SLAEEEIVIRSKNLRDNAKIIIVQLNKSVEIVCTRPNNGGSGSGGDIRQ
AYCNISGRNWSEAVNQVKKKLKEHFPHKNISFQSSSGGDLEITTHSFNC
GGEFFYCNTSGLFNDTISNATIMLPCRIKQIINMWQEVGKAIYAPPIKG
NITCKSDITGLLLLRDGGDTTDNTEIFRPSGGDMRDNWRSELYKYKVVE
IKPL.

Particular embodiments of the engineered Env include the following mutations: N460D; N463D; S278R; G471S; V65C; S115C; removal of V1 and V2; V3 replacement with a flexible linker; and an N-terminal truncation and a C-terminal truncation. In particular forms of these embodiments, the engineered Env does not include a mutation at position 276.

Particular embodiments of the engineered Env include the following mutations: N460D; N463D; S278R; and G471S; removal of V1 and V2; V3 replacement with a flexible linker; and an N-terminal truncation and a C-terminal truncation. In particular forms of these embodiments, the engineered Env does not include a mutation at position 276. In particular embodiments, V65C and S115C can optionally be included to stabilize the Env following removal of the V1 and V2 loops.

Particular embodiments of the engineered Env include the following mutations: N460D; N463D; S278R; G471S; V65C; S115C; removal of V1 and V2; V3 replacement with a flexible linker; and an N-terminal truncation. In particular embodiments, V1 refers to 131-152 and V2 refers to 161-196. In particular embodiments, removal of V1 and V2 loops includes removal of 123-196. In particular embodiments, V3 refers to 296-331. In particular embodiments, removal of V3 with a flexible linker replacement includes removal of 301-323 and replacement with GGSGSG (SEQ ID NO: 2). Particular embodiments exclude a mutation at position 276. In the presence of the S278R mutation, the unmutated 276 position is not glycosylated. Exclusion of a mutation at this position was unexpected because as previously stated, this position is an important NLGS site used by HIV to avoid B cell detection. Particular embodiments disclosed herein present the outer domain and the inner domain.

In addition to the Gly-Ser linker of SEQ ID NO: 2, a number of flexible linkers can be used. In particular embodiments, a flexible linker is used to replace V3. The linker sequence should not be significantly deleterious to the immunogenicity of the engineered Env and may even be beneficial to immunogenicity. Such linkers are known to those of skill in the art. One exemplary flexible linker includes Ac-Cys-Gly-Gly-Gly (SEQ ID NO: 3). Additional flexible linkers include GSTSGSGKPGSGEGSTKG (SEQ ID NO: 4) and SGRAHAG (SEQ ID NO: 5). Further examples include a linker that includes (Gly)ₙ, where n=1 to 10 (e.g., n=1, 2, 3, 4 (SEQ ID NO: 6), 5 (SEQ ID NO: 7), 6 (SEQ ID NO: 8), 7 (SEQ ID NO: 9), 8 (SEQ ID NO: 10), 9 (SEQ ID NO: 11), or 10 (SEQ ID NO: 12); (Ser)ₙ, where n=1 to 10 (e.g., n=1, 2, 3, 4 (SEQ ID NO: 13), 5 (SEQ ID NO: 14), 6 (SEQ ID NO: 15), 7 (SEQ ID NO: 16), 8 (SEQ ID NO: 17), 9 (SEQ ID NO: 18), or 10 (SEQ ID NO: 19); (Ala)ₙ, where n=1 to 10 (e.g., n=1, 2, 3, 4 (SEQ ID NO: 20), 5 (SEQ ID NO: 21), 6 (SEQ ID NO: 22), 7 (SEQ ID NO: 23), 8 (SEQ ID NO: 24), 9 (SEQ ID NO: 25), or 10 (SEQ ID NO: 26); (Gly-Ser)ₙ, where n=1, 2 (SEQ ID NO: 27), 3 (SEQ ID NO: 28), 4 (SEQ ID NO: 29), 5 (SEQ ID NO: 30), 6 (SEQ ID NO: 31), 7 (SEQ ID NO: 32), 8 (SEQ ID NO: 33), 9 (SEQ ID NO: 34), or 10 (SEQ ID NO: 35); (Gly-Ser-Ser-Gly)ₙ, where n=1 (SEQ ID NO: 36), 2 (SEQ ID NO: 37), 3 (SEQ ID NO: 38), 4 (SEQ ID NO: 39), 5 (SEQ ID NO: 40), 6 (SEQ ID NO: 41), 7 (SEQ ID NO: 42), 8 (SEQ ID NO: 43), 9 (SEQ ID NO: 44), or 10 (SEQ ID NO: 45); (Gly-Ser-Gly)ₙ, where n=1, 2 (SEQ ID NO: 46), 3 (SEQ ID NO: 47), 4 (SEQ ID NO: 48), 5 (SEQ ID NO: 49), 6 (SEQ ID NO: 50), 7 (SEQ ID NO: 51), 8 (SEQ ID NO: 52), 9 (SEQ ID NO: 53), or 10 (SEQ ID NO: 54); (Gly-Ser-Ser)ₙ, where n=1, 2 (SEQ ID NO: 55), 3 (SEQ ID NO: 56), 4 (SEQ ID NO: 57), 5 (SEQ ID NO: 58), 6 (SEQ ID NO: 59), 7 (SEQ ID NO: 60), 8 (SEQ ID NO: 61), 9 (SEQ ID NO: 62), or 10 (SEQ ID NO: 63); (Gly-Ala)ₙ, where n=1, 2 (SEQ ID NO: 64), 3 (SEQ ID NO: 65), 4 (SEQ ID NO: 66), 5 (SEQ ID NO: 67), 6 (SEQ ID NO: 68), 7 (SEQ ID NO: 69), 8 (SEQ ID NO: 70), 9 (SEQ ID NO: 71), or 10 (SEQ ID NO: 72); or any combination thereof.

An N-terminal truncation refers to a truncation at the N-terminal end of a naturally-occurring Env. In particular embodiments, the N-terminal truncation is before residue 49, 48, 47, 46, 45, 44, 43, 42, 41, 40 or 39. In particular embodiments, the N-terminal truncation is before residue 46, 45, 44, 43 or 42. In particular embodiments, the N-terminal truncation is before residue 44.

Particular embodiments include a C-terminal truncation. In particular embodiments, the C-terminal truncation is after residue 499, 498, 497, 496, 495, 494, 493, 492, 491, 490 or 389. In particular embodiments, the C-terminal truncation is after residue 496, 495, 494, 493 or 492. In particular embodiments, the C-terminal truncation is after residue 494.

In particular embodiments, the key mutation on the 426c core that is required for glVRC01 binding knocks-out N276. In particular embodiments, the NLGS at position N460 should be eliminated. In particular embodiments, the N463 on the 426c core may be retained. In particular embodiments, the glycans at N463 on the 426c core can stabilize the binding of glVRC01 to the 426c core that lacks N276. One reason why glVRC01 binds the 426c core once N276 is knocked out is because the 426c Env naturally lacks a conserved NLGS at position 234. It is possible that N234, when glycosylated, may block the binding of glVRC01 even when N276 is knocked out.

Particular engineered Env sequences useful within the present disclosure also include those that (i) maintain high affinity for broadly neutralizing VRC01 class antibodies; (ii) bind with little or no detectable affinity to non-neutralizing CD4 binding site (bs) antibodies such as b6, b13, F105, 15e, m14 or m18; (iii) lack the V3 loop and beta20/21 hairpin and are minimal in size (175 residues compared to 230 for wild-type outer domain); (iv) display no or low evidence of aggregation; (v) have N and C termini located distal from the CD4bs to allow coupling, by chemical or genetic means, to larger particles for the purpose of multimeric display; and/or (vi) may be expressed with a minimum of only two glycans which may be useful for manipulating immune responses.

In particular embodiments, high affinity means that a binding domain associates with its target epitope with a dissociation constant (K_{D}) of 10⁵ M or less, in one embodiment of from 10-5 M to 10⁻¹³ M, or in one embodiment of from 10⁻⁵ M to 10⁻¹⁰ M. In particular embodiments, high affinity means that a binding domain associates with its target epitope with a dissociation constant (K_{D}) of 10⁻⁷ M or less, or in one embodiment of from 10⁻⁷ M to 10⁻¹² M, or in one embodiment of from 10⁻⁷ M to 10⁻¹⁵ M.

In particular embodiments, little or no detectable affinity means that the binding domain associates with its target epitope with a dissociation constant (K_{D}) of 10⁻⁴ M or more, in one embodiment of from 10⁻⁴ M to 1 M.

Exemplary engineered Env that bind glBCR and can be used in the immunization strategies disclosed herein include:

| SEQ ID | |
|---|---|
| NO. | Sequence |
| 73 | RPVVSTQLLLNGSLAEEEVVIRSVNFTDNAKTIIVQLNTSVEINCTGAGHCNISRAK |
| | WNNTLKQIASKLREQFGNNKTIIFKQSSGGDPEIVTHSFNCGGEFFYCDSTQLFNS |
| | TWFNSTWSTEGSNNTEGSDTITLPCRPAPPPHCSSNITGLLLTRDGGNSNNESEIF |
| | RPGGGDMRDNWRSE |
| 74 | RPVVSTQLLLNGSLAEEEVVIRSVNFTDNAKTIIVQLNTSVEINCTGAGHCNISRAK |
| | WNNTLKQIASKLREQFGNNKTIIFKQSSGGDPEIVTHSFNCGGEFFYCNSTQLFNS |
| | TWFNSTWSTEGSNNTEGSDTITLPCRPAPPPHCSSNITGLLLTRDGGNSNNESEIF |
| | RPGGGDMRDNWRSE |
| 75 | DTITLPCRPAPPPHCSSNITGLLLTRDGGNSNNESEIFRPGGGDMRDNWRSGLSG |
| | PVVSTQLLLNGSLAEEEVVIRSVNFTDNAKTIIVQLNTSVEINCTGAGHCNISRAKW |
| | NNTLKQIASKLREQFGNNKTIIFKQSSGGDPEIVTHSFNCGGEFFYCDSTQLFNST |
| | WFNSTWS |
| 76 | GEFFYCDSTQLFNSTWFNSTWSTEGSNNTEGSDTITLPCRPAPPPHCSSNITGLL |
| | LTRDGGNSNNESEIFRPGGGDMRDNWRSGLSGPVVSTQLLLNGSLAEEEVVIRS |
| | VNFTDNAKTIIVQLNTSVEINCTGAGHCNISRAKWNNTLKQIASKLREQFGNNKTIIF |
| | KQSSGGDPEIVTHSFNCG |
| 77 | DTITLPCRPAPPPHCSSNITGLILTRDGGNSNNESEIFRPGGGDMRDIARCQIAGTV |
| | VSTQLLLNGSLAEEEVVIRSVNFTDNAKSICVQLNTSVEINCTGAGHCNISRAKWN |
| | NTLKQIASKLREQFGNNKTIIFKQSSGGDPEIVTHSFNCGGEFFYCDSTQLFNSTW |
| | FNSTWS |
| 78 | DTITLPCRPAPPPHCSSNITGLILTRDGGNSNNESEIFRPGGGDMRDIARCQIAGTV |
| | VSTQLLLNGSLAEEEVVCRSVNFTDNAKSICVQLNTSVEINCTGAGHCNISRAKWN |
| | NTLKQIASCLREQFGNNKTIIFKQSSGGDPEIVTHSFNCGGEFFYCDSTQLFNSTW |
| | FNSTWS |
| 79 | DTITLPCRPAPPPHCSSNITGLILTRDGGNSNNESEIFRPGGGDMAGMPRCGGGA |
| | VSTQLLLNGSLAEEEVVCRSVNFTDNAKSICVQLNTSVEINCTGAGHCNISRAKWN |
| | NTLKQIASCLREQFGNNKTIIFKQSSGGDPEIVTHSFNCGGEFFYCDSTQLFNSTW |
| | F |
| 80 | DTITLPCRPAPPPHCSSNITGLILTRDGGNSNDESEIFRPGGGDMRDIARCQIAGTV |
| | VSTQLLLNGSLAEEEVVIRSVDFTDNAKSICVQLDTSVEIDCTGAGHCDISRAKWD |
| | NTLKQIASKLREQFGNDKTIIFKQSSGGDPEIVTHSFNCGGEFFYCDSTQLFDSTW |
| | FDSTWS |
| 81 | GEFFYCDSTQLFNSTWFNSTWSTEGSNNTEGSDTITLPCRPAPPPHCSSNITGLIL |
| | TRDGGNSNNESEIFRPGGGDMRDIARCQIAGTVVSTQLLLNGSLAEEEVVIRSVNF |
| | TDNAKSICVQLNTSVEINCTGAGHCNISRAKWNNTLKQIASKLREQFGNNKTIIFKQ |
| | SSGGDPEIVTHSFNCG |
| 82 | GEFFYCDSTQLFNSTWFNSTWSTEGSNNTEGSDTITLPCRPAPPPHCSSNITGLIL |
| | TRDGGNSNNESEIFRPGGGDMRCGARSGIAGTVVSTQLLLNGSLAEEEVVIRSVN |
| | FTDNAKCIIVQLNTSVEINCTGAGHCNISRAKWNNTLKQIASKLREQFGNNKTIIFKQ |
| | SSGGDPEIVTHSFNCG |
| 83 | GEFFYCDSTQLFNSTWFNSTWSTEGSNNTEGSDTITLPCRPAPPPHCSSNITGLIL |
| | TRDGGNSNNESEIFRPGGGDMRDIARCQIAGTVVSTQLLLNGSLAEEEVVCRSVN |
| | FTDNAKSICVQLNTSVEINCTGAGHCNISRAKWNNTLKQIASCLREQFGNNKTIIFK |
| | QSSGGDPEIVTHSFNCG |
| 84 | DTITLPCRPAPPPHCSSNITGLILTRDGGTSDDKTEIFRPGGGDMRDIARCQIAGTV |
| | VSTQLLLNGSLAEEEVVIRSEDFRDNAKSICVQLNTSVEINCTGAGHCNISRAKWN |
| | NTLKQIASKLREQFGNKTIIFKQSSGGDPEFVTHSFNCGGEFFYCDSTQLFDSTWF |
| | DSTWS |
| 85 | DTITLPCRPAPPPHCSSNITGLILTRGGGISDDKTEIFRPGGGDMRDIARCQIAGTV |
| | VSTQLLLNGSLAEEEVVIRSEDFRDNSKSICVQLNTSVEINCTGAGHCNISRAKWN |
| | NTLKQIASKLREQFGNKTIIFSQSLGGDPEFVTHSFNCGGEFFYCDSTQLFDSTWF |
| | DSTWS |
| 86 | DTITLPCRPAPPPHCSSNITGLILTRGGGISDDKTEIFRPGGGDMRDIARCQIAGTV |
| | VSTQLLLNGSLAEEEVVIRSENFTDNSKSICVQLNTSVEINCTGAGHCNISRAKWN |
| | NTLKQIASKLREQFGNKTIIFSQSLGGDPEFVTHSFNCGGEFFYCDSTQLFDSTWF |
| | DSTWS |
| 87 | DTITLPCRPAPPPHCSSNITGLILTRGGGISDDKTEIFRPSGGDMRDIARCQIAGTV |
| | VSTQLLLNGSLAEEEVVIRSEDFRDNSKSICVQLNTSVEINCTGAGHCNISRAKWN |
| | NTLKQIASKLREQFGNKTIIFSQSLGGDPEFVTHSFNCGGEFFYCDSTQLFDSTWF |
| | DSTW |
| 88 | DTITLPCRPAPPPHCSSNITGLILTRGGGISDDKTEIFRPGGGDMRDIARCQIAGTV |
| | VSTQLLLNGSLAEEEVVIRSEDFRDNSKSICVQLNTSVEINCTGAGHCNISRAKWN |
| | NTLKQIASKLREQFGNRTIIFSQSLGGDPEFVTHSFNCGGEFFYCDSTQLFDSTWF |
| | DSTW |
| 89 | DTITLPCRPAPPPHCSSNITGLILTRGGGISDDDTEIFRPSGGDMRDIARCQIAGTV |
| | VSTQLFLNGSLAEEEVVIRSEDFRDNSKSICVQLNTSVEINCTGAGHCNISRAKWN |
| | NTLKQIASKLREQFGNRTIIFSQSLGGDPEFVTHSFNCGGEFFYCDSTQLFDSTWF |
| | DSTW |
| 90 | DTITLPCRPAPPPHCSSNITGLILTRAGGISDDNTEIFRPSGGDMRDIARCQIAGTVV |
| | STQLFLNGSLAEEEVVIRSEDFRDNSKSICVQLNTSVEINCTGAGHCNISRAKWNN |
| | TLKQIASKLREQFGNRTIIFSQSLGGDPEFVTHSFNCGGEFFYCDSTQLFDSTWFD |
| | STW |
| 91 | DTITLPCRPAPPPHCSSNITGLILTRGGGISDDNTEIFRPSGGDMRDIARCQIAGTV |
| | VSTQLLLNGSLAEEEVVIRSEDFRDNSKSICVQLNTSVEINCTGAGHCNISRAKWN |
| | NTLKQIASKLREQFGNRTIIFSQSLGGDPEFVTHSFNCGGEFFYCDSTQLFDSTWF |
| | DSTW |
| 92 | DTITLPCRPAPPPHCSSNITGLILTRGGGVSDDDTEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFSQSSGGDPEFVTHSFNCGGEFFYCDSTQLFDSTWF |
| | DSTW |
| 93 | DTITLPCRPAPPPHCSSNITGLILTRGGGVSDDDTEIFRPAGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFSQSSGGDPEFVTHSFNCGGEFFYCDSTQLFDSTWF |
| | DSTW |
| 94 | DTITLPCRPAPPPHCSSNITGLILTRGGGVSDDDTEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFSQSSGGDPEFVTHSFNCGGEFFYCDSTQLFDSTWF |
| | D |
| 95 | DTITLPCRPAPPPHCSSNITGLILTRDGGVSNDETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCDSTQLFNSTWF |
| | NST |
| 96 | DTITLPCRPAPPPHCSSNITGLILTRDGGNSNDETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCDSTQLFNSTWF |
| | NST |
| 97 | DTITLPCRPAPPPHCSSNITGLILTRDGGVSNDESEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCDSTQLFNSTWF |
| | NST |
| 98 | DTITLPCRPAPPPHCSSNITGLILTRDGGVSNDETEIFRPGGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCDSTQLFNSTWF |
| | NST |
| 99 | DTITLPCRPAPPPHCSSNITGLILTRDGGVSNDETEIFRPSGGDMRDIARCQIAGTV |
| | VSTQLLLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCDSTQLFNSTWF |
| | NST |
| 100 | DTITLPCRPAPPPHCSSNITGLILTRDGGVSNDETEIFRPSGGDMRDIARCQIAGTV |
| | VSTQLFLNGSLAEEEVVIRSVDFTDNAKSICVQLNTSVEINCTGAGHCNISRAKWN |
| | NTLKQIASKLREQFGNRTIIFKQSSGGDPEFVTHSFNCGGEFFYCDSTQLFNSTWF |
| | NST |
| 101 | DTITLPCRPAPPPHCSSNITGLILTRDGGVSNDETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNKTIIFKQSSGGDPEFVTHSFNCGGEFFYCDSTQLFNSTWF |
| | NST |
| 102 | DTITLPCRPAPPPHCSSNITGLILTRDGGVSNDETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEIVTHSFNCGGEFFYCDSTQLFNSTWF |
| | NST |
| 103 | DTITLPCRPAPPPHCSSNITGLILTRDGGVSNDETEIFRPSGGDMRDIARCQIAGTV |
| | VSTQLFLNGSLAEEEVVIRSVDFRDNAKSICVQLDTSVEIDCTGAGHCDISRAKWD |
| | NTLKQIASKLREQFGDRTIIFKQSSGGDPEFVTHSFNCGGEFFYCDSTQLFDSTWF |
| | DST |
| 104 | DTITLPCRPAPPPHCSSNITGLILTRDGGVSNNETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCNSTQLFNSTWF |
| | NST |
| 105 | DTITLPCRPAPPPHCSSNITGLILTRDGGNSNNETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCNSTQLFNSTWF |
| | NST |
| 106 | DTITLPCRPAPPPHCSSNITGLILTRDGGVSNNETEIFRPGGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCNSTQLFNSTWF |
| | NST |
| 107 | DTITLPCRPAPPPHCSSNITGLILTRDGGVSNNETEIFRPSGGDMRDIARCQIAGTV |
| | VSTQLLLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCNSTQLFNSTWF |
| | NST |
| 108 | DTITLPCRPAPPPHCSSNITGLILTRDGGVSNNETEIFRPSGGDMRDIARCQIAGTV |
| | VSTQLFLNGSLAEEEVVIRSVDFTDNAKSICVQLNTSVEINCTGAGHCNISRAKWN |
| | NTLKQIASKLREQFGNRTIIFKQSSGGDPEFVTHSFNCGGEFFYCNSTQLFNSTWF |
| | NST |
| 109 | DTITLPCRPAPPPHCSSNITGLILTRDGGVSNNETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNKTIIFKQSSGGDPEFVTHSFNCGGEFFYCNSTQLFNSTWF |
| | NST |
| 110 | DTITLPCRPAPPPHCSSNITGLILTRDGGVSNNETEIFRPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEIVTHSFNCGGEFFYCNSTQLFNSTWF |
| | NST |
| 111 | DTITLPCRPAPPPHCSSNITGLILTRDGGVSNNETEIFRPSGGDMRDIARCQIAGTV |
| | VSTQLFLNGSLAEEEVVIRSVNFTDNAKSICVQLNTSVEINCTGAGHCNISRAKWN |
| | NTLKQIASKLREQFGNRTIIFKQSSGGDPEFVTHSFNCGGEFFYCNSTQLFNSTWF |
| | NST |
| 112 | DTITLPCRPAPPPHCSSNITGLILTRAGGVSDNNTEIFFPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFSQSTGGDPEFVTHSFNCGGEFFYCNSTQLFNSTWF |
| | NST |
| 113 | DTITLPCRPAPPPHCSSNITGLILGRAGGASDDNTEIFYPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFSQSTGGDPEIVTHSFNCGGEFFYCNSTQLFNSTWF |
| | NSTW |
| 114 | DTITLPCRPAPPPHCSSNITGLILTRAGGVSNNETEIFFPSGGDMRDIARCQIAGTV |
| | VSTQLFLNGSLAEEEVVIRSVDFRDNAKSICVQLNTSVEINCTGAGHCNISRAKWN |
| | NTLKQIASKLREQFGNRTIIFKQSSGGDPEFVTHSFNCGGEFFYCNSTQLFNSTWF |
| | NST |
| 115 | VWKEATTTLFCASDAKAYDTEVHNVWATHACVPTDPNPQEVELENVTENFNMWK |
| | NNMVEQMHEDIISLWDQSLKPCVKLTGGSVITQACPKISFEPIPIHYCAPAGFAILK |
| | CKDKKFNGKGPCSNVSTVQCTHGIRPVVSTQLLLNGSLAEEEVVIRSENFADNAK |
| | TIIVQLNESVEINCTRPNNGGSGSGGDIRQAHCNLSRAKWNDTLNKIVIKLREQFG |
| | NKTIVFKHSSGGDPEIVTHSFNCGGEFFYCNSTQLFNSTWNVTEESNNTVENNTIT |
| | LPCRIKQIINMWQKVGRAMYAPPIRGQIRCSSNITGLLLTRDGGPEDNKTEVFRPG |
| | GGDMRDNWRSELYKYKVVKIE |
| 116 | VWKDATTTLFCASDAKAHETEVHNVWATHACVPTDPNPQEVELKNVTENFNMW |
| | KNNMVEQMHEDIISLWDQSLKPCVKLTGGSVITQACPKVSFEPIPIHYCAPAGFAIL |
| | KCKDKKFNGKGPCSNVSTVQCTHGIRPVVSTQLLLNGSLAEEEVVIRSEDFRNNA |
| | KIIIVQLNESVEINCTGAGHCNLSRAKWNDTLNKIVIKLREQFGNKTIVFKHSSGGDP |
| | EIVTHSFNCGGEFFYCNSTQLFNSTWNVTEESNNTVENNTITLPCRIKQIINMWQE |
| | VGRAMYAPPIRGQIRCSSNI |

Within these examples, SEQ ID NOs: 73-83 are advantageous for the elicitation of CD4-binding site (CD4bs)-directed broadly-neutralizing antibodies (bNAbs), while SEQ ID NOs. 84-116 are advantageous for improving binding to glVRC01 and/or other VI-11-2 antibodies.

As disclosed herein, HIV Env are administered in coordination with taking an HIV-infected subject off of ART such that viral rebound may guide the maturation of HIV Env activated gl B cells. In particular embodiments, removal of ART therapy occurs 2, 3, 4, 5, 6, 7, or 8 weeks after administration of the HIV Env. In particular embodiments, removal of ART therapy occurs 2, 3, or 4 weeks after administration of the HIV Env.

In certain examples, removal of ART therapy occurs prior to administration of the HIV Env (e.g., 1, 2, 3, 4, 5, 6, or 7 days before administration of the HIV Env). In certain examples, removal of ART therapy occurs after administration of the HIV Env (e.g., 2, 3, 4, 5, 6, or 7 days after administration of the HIV Env). In certain examples, removal of ART therapy occurs on the same day as administration of the HIV Env (e.g., within 24 hours of administration of the HIV Env). Particular embodiments can include more than one administration of an HIV Env.

In certain examples, removal of ART therapy occurs prior to administration of the HIV Env (e.g., 8, 9, 10, 11, 12, 13, 14, or 15 days before administration of the HIV Env). In certain examples, removal of ART therapy occurs after administration of the HIV Env (e.g., 8, 9, 10, 11, 12, 13, 14, 15, 16, 17, 18, 19, 20, 21, 22, 23, 24, 25, 16, 27, 28, 29, 30, 31, 32, 33, 34, 35, 36, 37, 38, 39, 40, 41, or 42) days after administration of the HIV Env). In certain examples, removal of ART therapy occurs after administration of the HIV Env (e.g., 14, 15, 16, 17, 18, 19, 20, 21, 22, 23, 24, 25, 16, 27, or 28) days after administration of the HIV Env).

In certain examples, removal of ART therapy occurs on the same day as administration of the HIV Env (e.g., within 24 hours of administration of the HIV Env).

Particular embodiments can include administration of more than one HIV Env and/or more than one administration of an HIV Env. For example, more than one HIV Env can be administered as a combination vaccine simultaneously. In other examples, a first administration of an HIV Env can be referred to as a prime immunization and a second administration of an HIV Env can be referred to as a boost administration. In certain examples, the boost administration will include the same HIV Env as administered during the prime immunization. In certain examples, the boost administration will include the same combination of HIV Env as administered during the prime immunization. In certain examples, the boost administration is a different HIV Env or a different combination of HIV Env than administered during the prime immunization. In certain examples, the boost immunization will include an HIV Env with an NLGS at position 276.

A prime immunization and a boost administration can be administered with a clinically relevant delay between the prime immunization and the boost immunization. For example, the clinically relevant delay can be 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month, 5 weeks, 6 weeks, 7 weeks, 2 months, 3 months, 4 months, 5 months, or 6 months.

In certain examples, the removal of ART therapy is time-coordinated with the administration of a boost immunization. In particular embodiments, removal of ART therapy occurs 2, 3, 4, 5, 6, 7, or 8 weeks after administration of the boost immunization. In particular embodiments, removal of ART therapy occurs 2, 3, or 4 weeks after administration of the boost immunization.

In certain examples, removal of ART therapy occurs after administration of the boost immunization (e.g., 8, 9, 10, 11, 12, 13, 14, 15, 16, 17, 18, 19, 20, 21, 22, 23, 24, 25, 16, 27, 28, 29, 30, 31, 32, 33, 34, 35, 36, 37, 38, 39, 40, 41, or 42) days after administration of the boost immunization. In certain examples, removal of ART therapy occurs after administration of the boost immunization (e.g., 14, 15, 16, 17, 18, 19, 20, 21, 22, 23, 24, 25, 16, 27, or 28) days after administration of the boost immunization.

In additional examples, removal of ART therapy occurs prior to administration of the boost immunization (e.g., 1, 2, 3, 4, 5, 6, or 7 days before administration of the boost immunization). In certain examples, removal of ART therapy occurs after administration of the boost immunization (e.g., 2, 3, 4, 5, 6, or 7 days after administration of the boost immunization). In certain examples, removal of ART therapy occurs on the same day as administration of the boost immunization (e.g., within 24 hours of administration of the boost immunization). Particular embodiments can include more than one administration of a boost immunization, for example, 2, 3, 4, or 5 boost immunizations.

In certain examples, removal of ART therapy occurs prior to administration of the boost immunization (e.g., 8, 9, 10, 11, 12, 13, 14, or 15 days before administration of the boost immunization). In certain examples, removal of ART therapy occurs after administration of the boost immunization (e.g., 8, 9, 10, 11, 12, 13, 14, or 15 days after administration of the HIV Env).

(iv) Multimerization of Env. A multimerized engineered Env refers to an assembly of two or more Env. Multimerization can enhance the immunogenicity of administered Env. In particular embodiments, multimers include trimers, tetramers, and octamers using coiled-coil multimerization domains. Trimers, tetramers, octamers, 24mers, 60mers, 180mers, or other larger order-mers can be formed.

Particular embodiments can utilize ferritin as a multimerization domain. Ferritin is an iron storage protein found in almost all living organisms, and has been extensively studied and engineered for a number of biochemical/biomedical purposes (US 20090233377; Meldrum, et al. Science 257, 522-523 (1992); U.S 20110038025; Yamashita, Biochim Biophys Acta 1800, 846-857 (2010), including as a multimerizing vaccine platform for displaying peptide epitopes (US 20060251679 (2006); Li, et al. Industrial Biotechnology 2, 143-147 (2006)). Ferritin is particularly useful for multimerizing vaccine epitopes because of its self-assembly and multivalent presentation of the epitopes which induces stronger B cell responses than monovalent forms and induces T-cell independent antibody responses (Bachmann et al., Annual Review of Immunology 15, 235-270 (1997); Dintzis et al. Proceedings of the National Academy of Sciences of the United States of America 73, 3671-3675 (1976)). Furthermore, the molecular architecture of ferritin, which can include 24 subunits assembling into an octahedral cage with 432 symmetry, has the ability to display multimeric antigens on its surface.

Particular embodiments utilize a monomeric ferritin subunit protein linked to an Env. The monomeric ferritin subunit protein can include a domain that allows the fusion protein to self-assemble into particles. The monomeric ferritin subunit protein can be selected from a bacterial ferritin, a plant ferritin, an algal ferritin, an insect ferritin, a fungal ferritin, and a mammalian ferritin. In particular embodiments, the monomeric ferritin can be a monomeric subunit of a Helicobacter pylori ferritin protein.

In particular embodiments, ferritin proteins from different sources (e.g., species) can be fused to form hybrid ferritins or ferritin fusion sequences. An exemplary hybrid includes the Helicobacter pylori-bullfrog ferritin fusion protein described in Kanekiyo et al., (Cell. 2015 Aug. 27; 162(5): 1090-1100). In particular embodiments, ferritin can include any one of SEQ ID NOs. 117-120.

| SEQ ID | |
|---|---|
| NO: | Sequence |
| 117 | ESQVRQQFSKDIEKLLNEQVNKEMQSSNLYMSMSSWCYTHSLDGAGLFLFDHAA |
| | EEYEHAKKLIIFLNENNVPVQLTSISAPEHKFEGLTQIFQKAYEHEQHISESINNIVD |
| | HAIKSKDHATFNFLQWYVAEQHEEEVLFKDILDKIELIGNENHGLYLADQYVKGIAK |
| | SRKSGS |
| 118 | DIIKLLNEQVNKEMNSSNLYMSMSSWCYTHSLDGAGLFLFDHAAEEYEHAKKLIIF |
| | LNENNVPVQLTSISAPEHKFEGLTQIFQKAYEHEQHISESINNIVDHAIKSKDHATFN |
| | FLQWYVAEQHEEEVLFKDILDKIELIGNENHGLYLADQYVKGIAKSRKS |
| 119 | LNEQVNKEMNSSNLYMSMSSWCYTHSLDGAGLFLFDHAAEEYEHAKKLIIFLNEN |
| | NVPVQLTSISAPEHKFEGLTQIFQKAYEHEQHISESINNIVDHAIKSKDHATFNFLQ |
| | WYVAEQHEEEVLFKDILDKIELIGNENHGLYLADQYVKGIAKSRKS |
| 120 | DIIKLLNEQVNKEMQSSNLYMSMSSWCYTHSLDGAGLFLFDHAAEEYEHAKKLIIF |
| | LNENNVPVQLTSISAPEHKFEGLTQIFQKAYEHEQHISESINNIVDHAIKSKDHATFN |
| | FLQWYVAEQHEEEVLFKDILDKIELIGNENHGLYLADQYVKGIAKSRKS |

The following sequence provides an exemplary 426c core+linker+ferritin construct:

| SEQ ID | |
|---|---|
| NO: | Sequence |
| 121 | VWKEAKTTLFCASDAKAYEKECHNVWATHACVPTDPNPQEVVLENVTENFNMW |
| | KNDMVDQMQEDVISIWDQCLKPCVKLTNTSTLTQACPKVTFDPIPIHYCAPAGYAI |
| | LKCNNKTFNGKGPCNNVSTVQCTHGIKPVVSTQLLLNGSLAEEEIVIRSKNLRDNA |
| | KIIIVQLNKSVEIVCTRPNNGGSGSGGDIRQAYCNISGRNWSEAVNQVKKKLKEHF |
| | PHKNISFQSSSGGDLEITTHSFNCGGEFFYCNTSGLFNDTISNATIMLPCRIKQIINM |
| | WQEVGKAIYAPPIKGNITCKSDITGLLLLRDGGDTTDNTEIFRPSGGDMRDNWRSE |
| | LYKYKVVEIKPLGSGGSGESQVRQQFSKDIEKLLNEQVNKEMQSSNLYMSMSSW |
| | CYTHSLDGAGLFLFDHAAEEYEHAKKLIIFLNENNVPVQLTSISAPEHKFEGLTQIF |
| | QKAYEHEQHISESINNIVDHAIKSKDHATFNFLQWYVAEQHEEEVLFKDILDKIELIG |
| | NENHGLYLADQYVKGIAKSRKSGS |

In particular embodiments, Env can be multimerized with a C4b multimerization domain. C4 binding protein (C4b) is the major inhibitor of the classical complement and lectin pathway. The complement system is a major part of innate immunity and is the first line of defense against invading microorganisms. Orchestrated by more than 60 proteins, its major task is to discriminate between host cells and pathogens and to initiate immune responses when necessary. It also recognizes necrotic or apoptotic cells (Hofmeyer et al., Journal of Molecular Biology. 2013 Apr. 26; 425(8):1302-17).

Full-length native C4b includes seven a-chains linked together by a multimerization (i.e., heptamerization) domain at the C-terminus of the a-chains (Blom et al., (2004) Molecular Immunology 40:1333-1346). One of the a-chains can be replaced by a B-chain in humans. The wild-type C4b multimerization domain is 57 amino acid residues in humans and 54 amino acid residues in mice (Forbes et al., PLoS One. 2012; 7(9):e44943). It contains an amphipathic α-helix region, which is necessary and sufficient for heptamerization, as well as two cysteine residues which stabilize the structure (Kask et al., (2002) Biochemistry 41:9349-9357).

The sequences of a number of C4b domain proteins are available in the art. These include human C4b multimerization domains as well as a number of homologues of human C4b multimerization domains available in the art. There are two types of homologues: orthologues and paralogues. Orthologues are defined as homologous genes in different organisms, i.e. the genes share a common ancestor coincident with the speciation event that generated them. Paralogues are defined as homologous genes in the same organism derived from a gene, chromosome or genome duplication, i.e. the common ancestor of the genes occurred since the last speciation event.

GenBank® (United States Department of Health and Human Services) indicates mammalian C4b multimerization domain homologues in species including chimpanzees, rhesus monkeys, rabbits, rats, dogs, horses, mice, guinea pigs, pigs, chicken, and cattle. Further C4b multimerization domains may be identified by searching databases of DNA or protein sequences, using commonly available search programs such as BLAST® (National Library of Medicine, Bethesda, MD).

In particular embodiments, a C4b multimerization domain includes a sequence as set forth in any one of SEQ ID NOs. 122-154

| SEQ ID | |
|---|---|
| NO. | Sequence |
| 122 | SGRAHAGWETPEGCEQVLTGKRLMQCLPNPEDVKMALEVYKLSLEIEQLELQRDS |
| | ARQSTLDKELVPR |
| 123 | KKQGDADVCGEVAYIQSVVSDCHVPTAELRTLLEIRKLFLEIQKLKVELQGLSKE |
| 124 | ETPEGCEQVLTGKRLMQCLPNPEDVKMALEVYKLSLEIEQLELQRDSARQSTLDKE |
| | L |
| 125 | WETPEGCEQVLTGKRLMQCLPNPEDVKMALEVYKLSLEIEQLELQRDSARQSTLD |
| | KEL |
| 126 | CEQVLTGKRLMQCLPNPEDVKMALEVYKLSLEIEQLELQRDSARQSTLDKEL |
| 127 | ETPEGCEQVLTGKRLMQCLPNPEDVKMALEVYKLSLEIEQLELQRDSARQYTLDKE |
| | L |
| 128 | ETPEGCEQVLAGKRLMQCLPNPEDVKMALEVYKLSLEIEQLELQRDRARQSTLDK |
| | EL |
| 129 | ETPEGCEQVLAGKRLMQCLPNPEDVKMALEVYKLSLEIEQLELQRDRARQSTWDK |
| | EL |
| 130 | EVPEGCEQVQAGRRLMQCLADPYEVKMALEVYKLSLEIELLELQRDKARKSSVLR |
| | QL |
| 131 | VVPEGCEHILKGRKTMQCLPNPEDVKMALEIYKLSLDIELLELQRDRAKESTVQSPV |
| 132 | EVPKDCEHVFAGKKLMQCLPNSNDVKMALEVYKLTLEIKQLQLQIDKAKHVDREL |
| 133 | EYPEDCEQVHEGKKLMQCLPNLEEIKLALELYKLSLETKLLELQIDKEKKAKAKYSI |
| 134 | EYPEDCEQVHEGKKLMECLPTLEEIKLALALYKLSLETNLLELQIDKEKKAKAKYST |
| 135 | EIAEGCEQVLAGRKIMQCLPKPEDVRTALELYKLSLEIKQLEKKLEKEEKCTPEVQE |
| 136 | EYPEGCEQVVTGRKLLQCLSRPEEVKLALEVYKLSLEIEILQTNKLKKEAFLLREREK |
| | NVTCDFNPE |
| 137 | EYPEGCEQVVTGRKLLKCLSRPEEVKLALEVYKLSLEIALLELQIDKPKDAS |
| 138 | EVPENCEQVIVGKKLMKCLSNPDEAQMALQLYKLSLEAELLRLQIVKARQGS |
| 139 | EASEDLKPALTGNKTMQYVPNSHDVKMALEIYKLTLEVELLQLQIQKEKHTEAH |
| 140 | VSAEVCEAVFKGQKLLKCLPNAMEVKMALEVYKLSLEIEKLEQEKRKLEIA |
| 141 | EVPEECKQVAAGRKLLECLPNPSDVKMALEVYKLSLEIEQLEKEKYVKIQEKFSKKE |
| | MKQLTSALH |
| 142 | EVLEDCRIVSRGAQLLHCLSSPEDVHRALKVYKLFLEIERLEHQKEKWIQLHRKPQS |
| | MK |
| 143 | EGPEDCEIVNKGRQLLQCLSSPEDVQRALEVYKLSLEIERLEQQREKRTSVHRKAH |
| | YTKVDGP |
| 144 | EAPEGCEQVLTGRKLMQCLPSPEDVKVALEVYKLSLEIKQLEKERDKLMNTHQKFS |
| | EKEEMKDLFFP |
| 145 | EVPEGCEQVLTGKKLMQCLPNPEDVKMALEVYKLSLEIELLELQIDKARQGS |
| 146 | GCEQVLTGKRLMQCLPNPEDVKMALEVYKLSLEIEQLELQRDSARQS |
| 147 | ETPEGCEQVLTGKRLMQCLPNPEDVKMALEVYKLSLEIEQLELQRDSARQS |
| 148 | GSEQVLTGKRLMQSLPNPEDVKMALEVYKLSLEIEQLELQRDSARQSTLDKEL |
| 149 | ETPEGCEQVLTGKRLMQCLPNPEDVKMALEIYKLTLEIEQLELQRDSARQSTLDKE |
| | L |
| 150 | ETPEGCEQVLTGKRLMQCLPNPEDVKMALEIYKLSLEIKQLELQRDSARQSTLDKE |
| | L |
| 151 | EGCEQILTGKRLMQCLPDPEDVKMALEIYKLSLEIKQLELQRDRARQSTL |
| 152 | ETPEGCEQVLTGKRLMQCLPNPEDVKMALEVYKLSLEIKQLELQRDRARQSTLDKE |
| | L |
| 153 | EGCEQILTGKRLMQCLPNPEDVKMALEIYKLSLEIEQLELQRDRARQSTLDK |
| 154 | WETPEGCEQVLTGKRLMQCLPNPEDVKMALEVYKLSLEIEQLELQRDSARQSTLD |
| | KELVPR |

In particular embodiments, the C4b multimerization domain will be a multimerization domain which includes (i) glycine at position 12, (ii) alanine at position 28, (iii) leucines at positions 29, 34, 36, and/or 41; (iv) tyrosine at position 32; (v) lysine at position 33; and/or (vi) cysteines at positions 6 and 18. In particular embodiments, the C4b multimerization domain will be a multimerization domain which includes (i) glycine at position 12, (ii) alanine at position 28, (iii) leucines at positions 29, 34, 36, and 41; (iv) tyrosine at position 32; (v) lysine at position 33; and (vi) cysteines at positions 6 and 18.

C4b multimerization domains can include any of SEQ ID NOs: 122-154 with an N-terminal deletion of at least 1 consecutive amino acid residue(s) (e.g., at least 2, 3, 4, 5, 6, 7, 8, 9, 10 consecutive amino acid residues) in length. Additional embodiments can include a C-terminal deletion of at least 1 consecutive amino acid residue(s) (eg. at least 2, 3, 4, 5, 6, 7, 8, 9, 10 consecutive amino acid residues) in length.

Particular C4b multimerization domain embodiments will retain or will be modified to include at least 1 of the following residues: A6, E11, A13, D21, C22, P25, A27, E28, L29, R30, T31, L32, L33, E34, 135, K37, L38, L40, E41, 142, Q43, K44, L45, E48, L49, or Q50. Further embodiments will retain or will be modified to include A6, E11, A13, D21, C22, P25, A27, E28, L29, R30, T31, L32, L33, E34, 135, K37, L38, L40, E41, 142, Q43, K44, L45, E48, L49, and Q50. Particular C4b multimerization domain embodiments will include the amino acid sequence: AELR (SEQ ID NO: 155).

Particular embodiments can utilize a heptamerization domain such as:

| SEQ | |
|---|---|
| ID NO: | Sequence |
| 156 | AHAGWETPEGCEQVLTGKRLMQCLPNPEDVKMALEVYKLSLEIEQLELQRDSARQ |
| | STLDKEL (Human) |
| 157 | SKKQGDADVCGEVAYIQSVVSDCHVPTEDVKTLLEIRKLFLEIQKLKVELQGLSKE |
| | (Chicken) |
| 158 | SKKQGDADVCGEVAYIQSVVSDCHVPTAELRTLLEIRKLFLEIQKLKVELQGLSKE |
| | (Modified Chicken) |

Particular embodiments of engineered 426c core Envs, GS linkers, and C4b multimerization domains include:

| SEQ | |
|---|---|
| ID NO: | Sequence |
| 159 | VWKEAKTTLFCASDAKAYEKECHNVWATHACVPTDPNPQEVVLENVTENFNMWK |
| | NDMVDQMQEDVISIWDQCLKPCVKLTNTSTLTQACPKVTFDPIPIHYCAPAGYAILK |
| | CNNKTFNGKGPCNNVSTVQCTHGIKPVVSTQLLLNGSLAEEEIVIRSKNLRDNAKIII |
| | VQLNKSVEIVCTRPNNGGSGSGGDIRQAYCNISGRNWSEAVNQVKKKLKEHFPHK |
| | NISFQSSSGGDLEITTHSFNCGGEFFYCNTSGLFNDTISNATIMLPCRIKQIINMWQE |
| | VGKAIYAPPIKGNITCKSDITGLLLLRDGGDTTDNTEIFRPSGGDMRDNWRSELYKY |
| | KVVEIKPLSGSGRAHAGWETPEGCEQVLTGKRLMQCLPNPEDVKMALEVYKLSLEI |
| | EQLELQRDSARQSTLDKELVPR |
| 160 | VWKEAKTTLFCASDAKAYEKECHNVWATHACVPTDPNPQEVVLENVTENFNMWK |
| | NDMVDQMQEDVISIWDQCLKPCVKLTNTSTLTQACPKVTFDPIPIHYCAPAGYAILK |
| | CNNKTFNGKGPCNNVSTVQCTHGIKPVVSTQLLLNGSLAEEEIVIRSKNLRDNAKIII |
| | VQLNKSVEIVCTRPNNGGSGSGGDIRQAYCNISGRNWSEAVNQVKKKLKEHFPHK |
| | NISFQSSSGGDLEITTHSFNCGGEFFYCNTSGLFNDTISNATIMLPCRIKQIINMWQE |
| | VGKAIYAPPIKGNITCKSDITGLLLLRDGGDTTDNTEIFRPSGGDMRDNWRSELYKY |
| | KVVEIKPLGSSKKQGDADVCGEVAYIQSVVSDCHVPTAELRTLLEIRKLFLEIQKLKV |
| | ELQGLSKE |

These engineered Env include: the following mutations: N460D, N463D, S278R, G471S, V65C, S115C, removal of V1 and V2, V3 replacement with a flexible linker, an N-terminal truncation before 44, a C-terminal truncation after 494, and a C4b multimerization domain. This engineered Env excludes a mutation at position 276, but nonetheless lacks N276 glycosylation due to the S278R mutation.

(v) Vaccine Adjuvants. Vaccines are often administered with vaccine adjuvants. The term “adjuvant” refers to material that enhances the immune response to an antigen and is used herein in the customary use of the term. The precise mode of action is not understood for all adjuvants, but such lack of understanding does not prevent their clinical use for a wide variety of vaccines.

Exemplary vaccine adjuvants include any kind of Toll-like receptor ligand or combinations thereof (e.g. CpG, Cpg-28 (a TLR9 agonist), Polyriboinosinic polyribocytidylic acid (Poly(I:C)), Adjuplex (a biodegradable matrix of carbomer homopolymer-Carbopol-and nanoliposomes), α-galactoceramide, monophosphoryl-lipid A (MPLA), Motolimod (VTX-2337, a novel TLR8 agonist developed by VentiRx), IMO-2055 (EMD1201081), TMX-101 (imiquimod), MGN1703 (a TLR9 agonist), Ribi (a TLR4 agonist), G100 (a stabilized emulsion of the TLR4 agonist glucopyranosyl lipid A), GLA-LSQ (a Glucopyranosyl lipid adjuvant in a liposomal formulation with QS21), Entolimod (a derivative of Salmonella flagellin also known as CBLB502), Hiltonol (a TLR3 agonist), and Imiquimod), and/or inhibitors of heat-shock protein 90 (Hsp90), such as 17-DMAG (17-dimethylaminoethylamino-17-demethoxygeldanamycin).

In particular embodiments a squalene-based adjuvant can be used. Squalene is part of the group of molecules known as triterpenes, which are all hydrocarbons with 30 carbon molecules. Squalene can be derived from certain plant sources, such as rice bran, wheat germ, amaranth seeds, and olives, as well as from animal sources, such as shark liver oil. In particular embodiments, the squalene-based adjuvant is MF59® (Novartis, Basel, Switzerland). An example of a squalene-based adjuvant that is similar to MF59® but is designed for preclinical research use is Addavax™ (InvivoGen, San Diego, CA). MF59 has been FDA approved for use in an influenza vaccine, and studies indicate that it is safe for use during pregnancy (Tsai T, et al. Vaccine. 2010. 17:28(7):1877-80; Heikkinen T, et al. American Journal of Obstetrics and Gynecology. 2012. 207(3):177). In particular embodiments, squalene-based adjuvants can include 0.1%- 20% (v/v) squalene oil. In particular embodiments, squalene-based adjuvants can include 5% (v/v) squalene oil.

In particular embodiments the adjuvant alum can be used. Alum refers to a family of salts that contain two sulfate groups, a monovalent cation, and a trivalent metal, such as aluminum or chromium. Alum is an FDA approved adjuvant. In particular embodiments, vaccines can include alum in the amounts of 1-1000 μg/dose or 0.1 mg-10 mg/dose.

In particular embodiments, the adjuvant Vaxfectin® (Vical, Inc., San Diego, CA) can be used. Vaxfectin® is a cationic lipid based adjuvant.

In particular embodiments, one or more STING agonists are used as a vaccine adjuvant. “STING” is an abbreviation of “stimulator of interferon genes”, which is also known as “endoplasmic reticulum interferon stimulator (ERIS)”, “mediator of IRF3 activation (MITA)”, “MPYS” or “transmembrane protein 173 (TM173)”. STING is a transmembrane receptor protein and is encoded by the gene TMEM173 in human. Activation of STING leads to production of Type I interferons (e.g., IFN-α and IFN-β), via the IRF3 (interferon regulatory factor 3) pathway; and to production of pro-inflammatory cytokines (e.g., TNF-α and IL-Iβ), via the NF-κB pathway and/or the NLRP3 inflammasome. Particular examples of STING agonists include c-AIMP; (3′,2′)c-AIMP; (2′,2′)c-AIMP; (2′,3′)c-AIMP; c-AIMP(S); c-(dAMP-dIMP); c-(dAMP-2′FdIMP); c-(2′FdAMP-2′FdIMP); (2′,3′) c-(AMP-2′FdIMP); c-[2′FdAMP(S)-2′FdIMP(S)]; c-[2′FdAMP(S)-2′FdIMP(S)](POM)2; and DMXAA. Additional examples of STING agonists are described in WO2016/145102.

Other immune stimulants can also be used as vaccine adjuvants. Additional exemplary small molecule immune stimulants include TGF-B inhibitors, SHP-inhibitors, STAT-3 inhibitors, and/or STAT-5 inhibitors. Exemplary siRNA capable of down-regulating immune-suppressive signals or oncogenic pathways (such as kras) can be used and any plasmid DNA (such as minicircle DNA) encoding immune-stimulatory proteins can also be used.

(vi) Compositions. The Env (in monomer or multimerized form (i.e., “active ingredients”) can be provided as part of compositions formulated for administration to subjects with or without inclusion of an adjuvant in the composition.

In particular embodiments, active ingredients are provided as part of a composition that can include, for example, at least 0.1% w/v or w/w of active ingredient(s), at least 1% w/v or w/w of active ingredient(s), at least 10% w/v or w/w of active ingredient(s), at least 20% w/v or w/w of active ingredient(s), at least 30% w/v or w/w of active ingredient(s), at least 40% w/v or w/w of active ingredient(s), at least 50% w/v or w/w of active ingredient(s), at least 60% w/v or w/w of active ingredient(s), at least 70% w/v or w/w of active ingredient(s), at least 80% w/v or w/w of active ingredient(s), at least 90% w/v or w/w of active ingredient(s), at least 95% w/v or w/w of active ingredient(s), or at least 99% w/v or w/w of active ingredient(s).

The compositions disclosed herein can be formulated for administration by, for example, injection, inhalation, infusion, perfusion, lavage or ingestion. The compositions can further be formulated for, for example, intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous administration and more particularly by intravenous, intradermal, intraarterial, intranodal, intralymphatic, intraperitoneal, intralesional, intraprostatic, intravaginal, intrarectal, topical, intrathecal, intratumoral, intramuscular, intravesicular, oral and/or subcutaneous injection.

For injection, compositions can be formulated as aqueous solutions, such as in buffers including Hanks' solution, Ringer's solution, or physiological saline. The aqueous solutions can contain formulatory agents such as suspending, stabilizing and/or dispersing agents. Alternatively, the formulation can be in lyophilized and/or powder form for constitution with a suitable vehicle, e.g., sterile pyrogen-free water, before use.

For oral administration, the compositions can be formulated as tablets, pills, dragees, capsules, liquids, gels, syrups, slurries, suspensions and the like. For oral solid formulations such as, for example, powders, capsules and tablets, suitable excipients include binders (gum tragacanth, acacia, cornstarch, gelatin), fillers such as sugars, e.g. lactose, sucrose, mannitol and sorbitol; dicalcium phosphate, starch, magnesium stearate, sodium saccharine, cellulose, magnesium carbonate; cellulose preparations such as maize starch, wheat starch, rice starch, potato starch, gelatin, gum tragacanth, methyl cellulose, hydroxypropylmethyl-cellulose, sodium carboxy-methylcellulose, and/or polyvinylpyrrolidone (PVP); granulating agents; and binding agents. If desired, disintegrating agents can be added, such as corn starch, potato starch, alginic acid, cross-linked polyvinylpyrrolidone, agar, or alginic acid or a salt thereof such as sodium alginate. If desired, solid dosage forms can be sugar-coated or enteric-coated using standard techniques. Flavoring agents, such as peppermint, oil of wintergreen, cherry flavoring, orange flavoring, etc. can also be used.

For administration by inhalation, compositions can be formulated as aerosol sprays from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator may be formulated containing a powder mix of the therapeutic and a suitable powder base such as lactose or starch.

Any composition formulation disclosed herein can advantageously include any other pharmaceutically acceptable carriers which include those that do not produce significantly adverse, allergic or other untoward reactions that outweigh the benefit of administration, whether for research, prophylactic and/or therapeutic treatments. Exemplary pharmaceutically acceptable carriers and formulations are disclosed in Remington's Pharmaceutical Sciences, 18th Ed. Mack Printing Company, 1990. Moreover, formulations can be prepared to meet sterility, pyrogenicity, general safety and purity standards as required by United States FDA Office of Biological Standards and/or other relevant foreign regulatory agencies.

Exemplary generally used pharmaceutically acceptable carriers include any and all bulking agents or fillers, solvents or co-solvents, dispersion media, coatings, surfactants, antioxidants (e.g., ascorbic acid, methionine, vitamin E), preservatives, isotonic agents, absorption delaying agents, salts, stabilizers, buffering agents, chelating agents (e.g., EDTA), gels, binders, disintegration agents, and/or lubricants.

Exemplary buffering agents include citrate buffers, succinate buffers, tartrate buffers, fumarate buffers, gluconate buffers, oxalate buffers, lactate buffers, acetate buffers, phosphate buffers, histidine buffers and/or trimethylamine salts.

Exemplary preservatives include phenol, benzyl alcohol, meta-cresol, methyl paraben, propyl paraben, octadecyldimethylbenzyl ammonium chloride, benzalkonium halides, hexamethonium chloride, alkyl parabens such as methyl or propyl paraben, catechol, resorcinol, cyclohexanol and 3-pentanol.

Exemplary isotonic agents include polyhydric sugar alcohols including trihydric or higher sugar alcohols, such as glycerin, erythritol, arabitol, xylitol, sorbitol or mannitol.

Exemplary stabilizers include organic sugars, polyhydric sugar alcohols, polyethylene glycol, sulfur-containing reducing agents, amino acids, low molecular weight polypeptides, proteins, immunoglobulins, hydrophilic polymers or polysaccharides.

Compositions can also be formulated as depot preparations. Depot preparations can be formulated with suitable polymeric or hydrophobic materials (for example as an emulsion in an acceptable oil) or ion exchange resins, or as sparingly soluble derivatives, for example, as sparingly soluble salts.

Additionally, compositions can be formulated as sustained-release systems utilizing semipermeable matrices of solid polymers containing at least one active ingredient. Various sustained-release materials have been established and are well known by those of ordinary skill in the art. Sustained-release systems may, depending on their chemical nature, release active ingredients following administration for a few weeks up to over 100 days.

(vii) Kits. Combinations of active ingredients can also be provided as kits. Kits can include containers including one or more Env, engineered Env, and/or vaccine adjuvants described herein formulated individually, or in various combinations.

Kits can also include a notice in the form prescribed by a governmental agency regulating the manufacture, use, or sale of pharmaceuticals or biological products, which notice reflects approval by the agency of manufacture, use, or sale for human administration. The notice may state that the provided active ingredients can be administered to a subject. The kits can include further instructions for using the kit, for example, instructions regarding preparation of components for administration; proper disposal of related waste; and the like. The instructions can be in the form of printed instructions provided within the kit or the instructions can be printed on a portion of the kit itself. Instructions may be in the form of a sheet, pamphlet, brochure, CD-Rom, or computer-readable device, or can provide directions to instructions at a remote location, such as a website. In particular embodiments, kits can also include some or all of the necessary medical supplies needed to use the kit effectively, such as syringes, ampules, tubing, facemask, an injection cap, sponges, sterile adhesive strips, Chloraprep, gloves, and the like. Variations in contents of any of the kits described herein can be made. The instructions of the kit will direct use of the active ingredients to effectuate a new clinical use described herein.

(viii) Methods of Use. Once formed, the compositions are used in immunization strategies to guide the maturation of antibodies against HIV in HIV-infected subjects. In particular embodiments, the compositions elicit antibodies that recognize a full length Env protein. In particular embodiments, the compositions find use in the treatment of disease. “Treatment” refers to both therapeutic treatment and prophylactic treatment or preventative measures, wherein the object is to prevent, reduce the occurrence or severity of, or slow down or lessen a targeted pathologic condition or disorder. “Subjects” include those in need of treatment, such as, those with an HIV infection taking one or more antiviral medications to treat the HIV. Thus, in various exemplary embodiments, a subject can be a human subject. Other types of subjects include appropriate research animals.

In particular embodiments, compositions can be administered to a subject in a therapeutically effective amount. A “therapeutically effective amount” is an amount sufficient to produce a desired physiological effect and/or an amount capable of achieving a desired result, particularly for treatment of a disorder or disease condition, including reducing or eliminating one or more symptom of the disorder or disease or prevention or delaying the onset of at least one a disease symptom. Therapeutically effective amounts can provide therapeutic treatments and/or prophylactic treatments.

Particular uses of the compositions include use as vaccines. Vaccines increase the immunity of a subject against a particular disease. Therefore, “HIV vaccine” can refer to a treatment that increases the immunity of a subject against HIV. In particular embodiments, the vaccine initiates the elicitation of antibodies that can bind a full length Env in subjects infected with HIV who have been undergoing HIV therapies such as ART. In particular embodiments, a vaccine can be used to ameliorate a symptom associated with AIDS or HIV infection, such as a reduced T cell count.

In particular embodiments, an HIV vaccine is a therapeutically effective composition including one or more Env or engineered Env disclosed herein that induce an immune response in a subject against HIV. The skilled artisan will appreciate that the immune system generally is capable of producing an innate immune response and an adaptive immune response. An innate immune response generally can be characterized as not being substantially antigen specific and/or not generating immune memory. An adaptive immune response can be characterized as being substantially antigen specific, maturing over time (e.g., increasing affinity and/or avidity for antigen), and in general can produce immunologic memory. Even though these and other functional distinctions between innate and adaptive immunity can be discerned, the skilled artisan will appreciate that the innate and adaptive immune systems can be integrated and therefore can act in concert.

“Immune response” refers to a response of the immune system to an Env disclosed herein. In various exemplary embodiments, an immune response to an Env can be an innate and/or adaptive response. In some embodiments, an adaptive immune response can be a “primary immune response” which refers to an immune response occurring on the first exposure of a “naive” subject to an engineered Env that binds a glBCR (e.g., a glVRC01 BCR). For example, in the case of a primary antibody response, after a lag or latent period of from 3 to 14 days depending on, for example, the composition, dose, and subject, gl antibodies to the engineered HIV Env can be produced. Generally, IgM production lasts for several days followed by IgG production and the IgM response can decrease. Antibody production can terminate after several weeks but memory cells can be produced. In some embodiments, an adaptive immune response can be a “secondary immune response”, “anamnestic response,” or “booster response” which refer to the immune response occurring after a potential second and subsequent exposure of a subject to a boost Env. In particular embodiments, the boost Env has an NLGS at position 276.

In particular embodiments, an immune response against HIV will include antibody production against the gp120 domain of an engineered Env

Antibodies that result from the disclosed immunization strategies are induced by HIV Env and natural viral rebound following the removal of ART therapy according to the methods disclosed herein. In some embodiments, an antibody can bind to a gp120 domain of an engineered Env. In some embodiments, an elicited antibody binds to gp120 (i.e., an etiologic agent of HIV). Without being bound by theory, in some embodiments, the binding of an antibody can substantially neutralize or inactivate autologous HIV gp120. Thus, antibodies are capable of reducing or eliminating a pathologic effect of HIV. That is, the binding of antibodies to gp120 of HIV may decrease or eliminate HIV infectivity and/or virulence factor activity, including replication, synthesis, and/or toxicity. In particular embodiments, at least a 25% decrease of one of these parameters is required to determine that a dose provides a therapeutically effective amount.

The actual dose amount of HIV Env administered to a particular subject as well as the timing between HIV Env administration and ART discontinuation can be determined and adjusted by a physician, veterinarian, or researcher taking into account parameters such as physical and physiological factors including target, body weight, severity of infection, stage of infection, previous or concurrent therapeutic interventions, idiopathy of the subject, and route of administration.

For administration, therapeutically effective amounts (also referred to herein as doses) can be initially estimated based on results from in vitro assays and/or animal model studies. Exemplary doses include 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240 or 250 μg/kg body mass or mg/kg body mass although higher and/or lower doses can be used. The number of doses that can be administered as a function of time can be from 1, 2, 3, 4 or 5 doses over 1, 2, 3, 4, 5 or 6 weeks but can be increased or decreased depending at least in part on the immune status of a subject.

In particular embodiments, a composition can be administered initially, and thereafter maintained by further administration. For example, a composition can be administered by intravenous injection to bring blood levels to a suitable level. The subject's levels can then be maintained by an oral boost form, although other forms of administration, dependent upon the patient's condition, may be used. In the instance of a vaccine composition, the HIV Env may be administered as a single dose, followed by one or more booster doses. For example, booster doses may assist natural rebounded virus in guiding the maturation of elicited antibodies to broadly neutralizing antibodies to provide bNAbs protective against multiple clades of HIV.

The engineered Env can be prepared by expressing polynucleotide sequences in vectors or other expression vehicles in compatible prokaryotic or eukaryotic host cells using standard molecular biology methods (e.g., Sambrook et al. 1989, Molecular Cloning a Laboratory Manual, Second Edition, Cold Spring Harbor Laboratory Press, Cold Spring Harbor, N.Y.; incorporated herein by reference).

(ix) Exemplary Embodiments. The Exemplary Embodiments below are included to demonstrate particular embodiments of the disclosure. Those of ordinary skill in the art should recognize in light of the present disclosure that many changes can be made to the specific embodiments disclosed herein and still obtain a like or similar result without departing from the spirit and scope of the disclosure.
1. A method of eliciting antibodies that bind full length glycosylated human immunodeficiency virus (HIV) envelope protein (Env) in a subject in need thereof, the method including
   administering to the subject an HIV Env that binds germline (gl) B cell receptors (BCR); and
   ceasing an anti-viral therapy to the subject. thereby eliciting antibodies that bind full length glycosylated HIV Env.
2. A method of embodiment 1, wherein the ceasing of the anti-viral therapy is at least 2 weeks after the administering.
3. A method of embodiment 1, wherein the ceasing of the anti-viral therapy is within 8 weeks after the administering.
4. A method of embodiment 1, wherein the ceasing of the anti-viral therapy is 2, 3, 4, 5, 6, 7, or 8 weeks after the administering.
5. A method of embodiment 1, wherein the ceasing of the anti-viral therapy is 2, 3, or 4 weeks after the administering.
6. A method of embodiment 1, wherein the ceasing of the anti-viral therapy is 14, 15, 16, 17, 18, 19, 20, 21, 22, 23, 24, 25, 16, 27, 28, 29, 30, 31, 32, 33, 34, 35, 36, 37, 38, 39, 40, 41, or 42 days after the administering.
7. A method of embodiment 1, wherein the ceasing of the anti-viral therapy is 14, 15, 16, 17, 18, 19, 20, 21, 22, 23, 24, 25, 16, 27, or 28 days after the administering.
8. A method of embodiment 1, wherein the ceasing of the anti-viral therapy is within 15 days before or 15 days after the administering.
9. A method of embodiment 1, wherein the ceasing of the anti-viral therapy is within 10 days before or 10 days after the administering.
10. A method of embodiment 1, wherein the ceasing of the anti-viral therapy is within 7 days before or 7 days after the administering.
11. A method of embodiment 1, wherein the ceasing of the anti-viral therapy is within 3 days before or 3 days after the administering.
12. A method of embodiment 1, wherein the ceasing of the anti-viral therapy is within 24 hours before or 24 hours after the administering.
13. A method of any of embodiment 1-12, wherein the HIV Env includes the 426c core including the sequence as set forth in SEQ ID NO: 1, or a sequence having at least 95% or 98% sequence identity to the sequence as set forth in SEQ ID NO: 1 .
14. A method of any of embodiments 1-13, wherein the HIV Env includes: (i) mutations at one or more of: N460D; N463D; S278R; G471S; V65C; and S115C; (ii) removal of the V1 loop and the V2 loop; (iii) replacement of the V3 loop with a flexible linker; and (iv) an N-terminal truncation; wherein the HIV Env does not have a mutation at position 276.
15. A method of any of embodiments 1-14, wherein the HIV Env includes mutations at N460D; N463D; S278R; G471S; V65C; and S115C.
16. A method of embodiments 13 or 14, wherein the N-terminal truncation is before residue 49, 48, 47, 46, 45, 44, 43, 42, 41, 40 or 39.
17. A method of embodiments 13 or 14, wherein the N-terminal truncation is before residue 44.
18. A method of any of embodiments 1-11, wherein the HIV Env further includes a C-terminal truncation after residue 499, 498, 497, 496, 495, 494, 493, 492, 491, 490 or 389.
19. A method of embodiment 14, wherein the C-terminal truncation is after residue 494.
20. A method of any of embodiments 14-19, wherein the flexible linker includes a sequence as set forth in any one of SEQ ID NOs: 2-72.
21. A method of any of embodiments 14-20, wherein the V3 loop includes residues 296-331.
22. A method of any of embodiments 14-21, wherein removal of the V1 loop includes removal of residues 131-152 and/or removal of the V2 loop includes removal of residues 161-196.
23. A method of any of embodiments 14-22, wherein removal of the V1 loop and removal of the V2 loop includes removal of residues 123-196.
24. A method of any of embodiments 1-23, wherein the HIV Env retains glycosylation at N463 but lacks glycosylation at N276 and N460.
25. A method of any of embodiments 1-24, wherein the HIV Env retains glycosylation at position 463.
26. A method of any of embodiments 1-25, wherein the HIV Env includes a sequence as set forth in any one of SEQ ID NOs: 73-116.
27. A method of any of embodiments 1-26, wherein the HIV Env is multimerized.
28. A method of embodiment 27, wherein the HIV Env is multimerized with ferritin.
29. A method of embodiment 28, wherein the ferritin includes a sequence as set forth in any one of SEQ ID NOs: 117-120.
30. A method of any of embodiments 1-27, wherein the HIV Env is multimerized with C4b.
31. A method of embodiment 24, wherein the C4b includes a sequence as set forth in any one of SEQ ID NOs: 122-154.
32. A method of any of embodiments 1-27, wherein the HIV Env is multimerized into 5-mers, 6-mers, or 7-mers.
33. A method of any of embodiments 1-27, wherein the HIV Env is multimerized into heptamers.
34. A method of embodiments 32 or 33, wherein the multimerization domain includes a sequence as set forth in any one of SEQ ID NOs: 156-158.
35. A method of any of embodiments 1-27, wherein the HIV Env is multimerized into 24-mers.
36. A method of any of embodiments 1-27, wherein the HIV Env includes a 426c core+linker+ferritin construct.
37. A method of embodiment 36, wherein the HIV Env includes the sequence as set forth in SEQ ID NO: 121 or a sequence having at least 95% or 98% sequence identity to the sequence as set forth in SEQ ID NO: 121.
38. A method of any of embodiments 1-27, wherein the HIV Env includes a 426c core+linker+C4b construct.
39. A method of embodiment 38, wherein the HIV Env includes the sequence as set forth in SEQ ID NO: 159 or 160.
40. A method of any of embodiments 1-39, wherein the glBCR are glVRC01 BCR.
41. A method of any of embodiments 1-40, further including administering a boost immunization including an HIV Env.
42. A method of any of embodiments 1 or 13-41, wherein the ceasing of the anti-viral therapy is at least 2 weeks after the administering of the boost immunization.
43. A method of any of embodiments 1 or 13-41, wherein the ceasing of the anti-viral therapy is within 8 weeks after the administering of the boost immunization.
44. A method of any of embodiments 1 or 13-41, wherein the ceasing of the anti-viral therapy is 2, 3, 4, 5, 6, 7, or 8 weeks after the administering of the boost immunization.
45. A method of any of embodiments 1 or 13-41, wherein the ceasing of the anti-viral therapy is 2, 3, or 4 weeks after the administering of the boost immunization.
46. A method of any of embodiments 1 or 13-41, wherein the ceasing of the anti-viral therapy is 14, 15, 16, 17, 18, 19, 20, 21, 22, 23, 24, 25, 16, 27, 28, 29, 30, 31, 32, 33, 34, 35, 36, 37, 38, 39, 40, 41, or 42 days after the administering of the boost immunization.
47. A method of any of embodiments 1 or 13-41, wherein the ceasing of the anti-viral therapy is 14, 15, 16, 17, 18, 19, 20, 21, 22, 23, 24, 25, 16, 27, or 28 days after the administering of the boost immunization.
48. A method of any of embodiments 1 or 13-41, wherein the ceasing of the anti-viral therapy is within 15 days before or 15 days after the administering of the boost immunization.
49. A method of embodiment 1 or 13-41, wherein the ceasing of the anti-viral therapy is within 10 days before or 10 days after the administering of the boost immunization.
50. A method of embodiment 1 or 13-41, wherein the ceasing of the anti-viral therapy is within 7 days before or 7 days after the administering of the boost immunization.
51. A method of embodiment 1 or 13-41, wherein the ceasing of the anti-viral therapy is within 3 days before or 3 days after the administering of the boost immunization.
52. A method of embodiment 1 or 13-41, wherein the ceasing of the anti-viral therapy is within 24 hours before or 24 hours after the administering of the boost immunization.
53. A method of any of embodiments 41-52, wherein the boost immunization comprises an HIV Env with an NLGS at position 276.
54. A method of any of embodiments 41-53, wherein the boost immunization comprises a same HIV Env as administered according to any of embodiments 1 or 13-39.
55. A method of any of embodiments 41-53, wherein the boost immunization comprises a different HIV Env as administered according to any of embodiments 1 or 13-39.
56. A method of any of embodiments 41-55, wherein the time between the administering of any of embodiments 1-40 and the administering the boost immunization of any of embodiments 41-55 is 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month, 5 weeks, 6 weeks, 7 weeks, 2 months, 3 months, 4 months, 5 months, or 6 months.
57. A method of any of embodiments 41-55, wherein the time between the administering of any of embodiments 1-40 and the administering the boost immunization of any of embodiments 41-55 is 1, 2, or 3 months.
58. A method of any of embodiments 41-55, wherein the time between the administering of any of embodiments 1-40 and the administering the boost immunization of any of embodiments 41-55 is 1, 2, or 3 months after a re-initiation of an anti-viral therapy.
59. A method of any of embodiments 1-58, further including administering an adjuvant to the subject.
60. A method of embodiment 59, wherein the administering of the adjuvant is with the administering of the HIV-Env.
61. A method of embodiment 59 or 60, wherein the administering of the adjuvant is with the boost immunization.
62. A method of any of embodiments 59-61, wherein the adjuvant includes one or more of PolyIC, Adjuplex, Alum, Ribi, or GLA-LSQ.
63. A method of any of embodiments 1-62, wherein the subject in need thereof received an HIV vaccination before the administering.
64. A method of embodiment 63, wherein the subject in need thereof received the HIV vaccination before the subject contracted HIV.
65. A method of embodiment 63, wherein the subject in need thereof received the HIV vaccination after the subject contracted HIV.
66. A method of embodiment 63, wherein the subject in need thereof received the HIV vaccination before the subject contracted HIV, and received a second HIV vaccination after the subject contracted HIV and before the administering.
67. A method of any of embodiments 1-66, wherein the anti-viral therapy is an anti-retroviral therapy.
68. A method of any of embodiments 1-67, wherein the anti-viral therapy includes a nucleoside reverse transcriptase inhibitor, a non-nucleoside reverse transcriptase inhibitor, a protease inhibitor, a fusion inhibitor, a CCR5 antagonist, an integrase strand transfer inhibitor, an attachment inhibitor, a post-attachment inhibitor, a capsid inhibitor, a pharmacokinetic enhancer, or a combination treatment.
69. A method of embodiment 68,
   wherein the nucleoside reverse transcriptase inhibitor includes abacavir, emtricitabine, lamivudine, tenofovir disoproxil, fumarate, or zidovudine;
   wherein the non-nucleoside reverse transcriptase inhibitor includes doravirine, efavirenz, etravirine, nevirapine, or rilpivirine;
   wherein the protease inhibitor includes atazanavir, darunavir, fosamprenavir, ritonavir or tipranavir;
   wherein the fusion inhibitor includes enfuvirtide;
   wherein the CCR5 antagonist includes maraviroc;
   wherein the integrase strand transfer inhibitor includes cabotegravir, dolutegravir, or raltegravir; wherein the attachment inhibitor includes fostemsavir;
   wherein the post-attachment inhibitor includes ibalizumab;
   wherein the capsid inhibitor includes lenacapavir;
   wherein the pharmacokinetic enhancer includes cobicistat; or
   wherein the combination treatment includes abacavir and lamivudine; abacavir, dolutegravir, and lamivudine; abacavir, lamivudine, and zidovudine; atazanavir and cobicistat; bictegravir, emtricitabine, and tenofovir alafenamide; cabotegravir and rilpivirine; darunavir and cobicistat; darunavir, cobicistat, emtricitabine, and tenofovir alafenamide; dolutegravir and lamivudine; dolutegravir and rilpivirine; doravirine, lamivudine, and tenofovir disoproxil fumarate; efavirenz, emtricitabine, and tenofovir disoproxil fumarate; efavirenz, lamivudine, and tenofovir disoproxil fumarate; efavirenz, lamivudine, and tenofovir disoproxil fumarate; elvitegravir, cobicistat, emtricitabine, and tenofovir alafenamide; elvitegravir, cobicistat, emtricitabine, and tenofovir disoproxil fumarate; emtricitabine, rilpivirine, and tenofovir alafenamide; emtricitabine, rilpivirine, and tenofovir disoproxil fumarate; emtricitabine and tenofovir alafenamide; emtricitabine and tenofovir disoproxil fumarate; lamivudine and tenofovir disoproxil fumarate; lamivudine and zidovudine; or lopinavir and ritonavir.

(x) Closing Paragraphs. The nucleic acid and amino acid sequences provided herein are shown using letter abbreviations for nucleotide bases and amino acid residues, as defined in 37 C.F.R. § 1.831-1.835 and set forth in WIPO Standard ST.26 (implemented on Jul. 1, 2022). Only one strand of each nucleic acid sequence is shown, but the complementary strand is understood as included in embodiments where it would be appropriate.

Variants of the sequences disclosed and referenced herein are also included. Guidance in determining which amino acid residues can be substituted, inserted, or deleted without abolishing biological activity can be found using computer programs well known in the art, such as DNASTAR™ (Madison, Wisconsin) software. Preferably, amino acid changes in the protein variants disclosed herein are conservative amino acid changes, i.e., substitutions of similarly charged or uncharged amino acids. A conservative amino acid change involves substitution of one of a family of amino acids which are related in their side chains.

In a peptide or protein, suitable conservative substitutions of amino acids are known to those of skill in this art and generally can be made without altering a biological activity of a resulting molecule. Those of skill in this art recognize that, in general, single amino acid substitutions in non-essential regions of a polypeptide do not substantially alter biological activity (see, e.g., Watson et al. Molecular Biology of the Gene, 4th Edition, 1987, The Benjamin/Cummings Pub. Co., p. 224). Naturally occurring amino acids are generally divided into conservative substitution families as follows: Group 1: Alanine (Ala), Glycine (Gly), Serine (Ser), and Threonine (Thr); Group 2: (acidic): Aspartic acid (Asp), and Glutamic acid (Glu); Group 3: (acidic; also classified as polar, negatively charged residues and their amides): Asparagine (Asn), Glutamine (Gln), Asp, and Glu; Group 4: Gln and Asn; Group 5: (basic; also classified as polar, positively charged residues): Arginine (Arg), Lysine (Lys), and Histidine (His); Group 6 (large aliphatic, nonpolar residues): Isoleucine (Ile), Leucine (Leu), Methionine (Met), Valine (Val) and Cysteine (Cys); Group 7 (uncharged polar): Tyrosine (Tyr), Gly, Asn, Gln, Cys, Ser, and Thr; Group 8 (large aromatic residues): Phenylalanine (Phe), Tryptophan (Trp), and Tyr; Group 9 (non-polar): Proline (Pro), Ala, Val, Leu, Ole, Phe, Met, and Trp; Group 11 (aliphatic): Gly, Ala, Val, Leu, and Ole; Group 10 (small aliphatic, nonpolar or slightly polar residues): Ala, Ser, Thr, Pro, and Gly; and Group 12 (sulfur-containing): Met and Cys. Additional information can be found in Creighton (1984) Proteins, W.H. Freeman and Company.

In making such changes, the hydropathic index of amino acids may be considered. The importance of the hydropathic amino acid index in conferring interactive biologic function on a protein is generally understood in the art (Kyte and Doolittle, 1982, J. Mol. Biol. 157(1), 105-32). Each amino acid has been assigned a hydropathic index on the basis of its hydrophobicity and charge characteristics (Kyte and Doolittle, 1982). These values are: Ile (+4.5); Val (+4.2); Leu (+3.8); Phe (+2.8); Cys (+2.5); Met (+1.9); Ala (+1.8); Gly (−0.4); Thr (−0.7); Ser (−0.8); Trp (−0.9); Tyr (−1.3); Pro (−1.6); His (−3.2); Glutamate (−3.5); Gln (−3.5); aspartate (−3.5); Asn (−3.5); Lys (−3.9); and Arg (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity.

As detailed in U.S. Pat. No. 4,554,101, the following hydrophilicity values have been assigned to amino acid residues: Arg (+3.0); Lys (+3.0); aspartate (+3.0±1); glutamate (+3.0±1); Ser (+0.3); Asn (+0.2); Gln (+0.2); Gly (0); Thr (−0.4); Pro (−0.5±1); Ala (−0.5); His (−0.5); Cys (−1.0); Met (−1.3); Val (−1.5); Leu (−1.8); Ile (−1.8); Tyr (−2.3); Phe (−2.5); Trp (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions may be based on the relative similarity of the amino acid side-chain substituents, for example, their hydrophobicity, hydrophilicity, charge, size, and the like.

As indicated elsewhere, variants of gene sequences can include codon optimized variants, sequence polymorphisms, splice variants, and/or mutations that do not affect the function of an encoded product to a statistically-significant degree.

Variants of the protein, nucleic acid, and gene sequences disclosed herein also include sequences with at least 70% sequence identity, at least 80% sequence identity, at least 85% sequence, at least 90% sequence identity, at least 95% sequence identity, at least 96% sequence identity, at least 97% sequence identity, at least 98% sequence identity, or at least 99% sequence identity to the protein, nucleic acid, or gene sequences disclosed herein.

“% sequence identity” refers to a relationship between two or more sequences, as determined by comparing the sequences. In the art, “identity” also means the degree of sequence relatedness between protein, nucleic acid, or gene sequences as determined by the match between strings of such sequences. “Identity” (often referred to as “similarity”) can be readily calculated by known methods, including those described in: Computational Molecular Biology (Lesk, A. M., ed.) Oxford University Press, NY (1988); Biocomputing: Informatics and Genome Projects (Smith, D. W., ed.) Academic Press, NY (1994); Computer Analysis of Sequence Data, Part I (Griffin, A. M., and Griffin, H. G., eds.) Humana Press, NJ (1994); Sequence Analysis in Molecular Biology (Von Heijne, G., ed.) Academic Press (1987); and Sequence Analysis Primer (Gribskov, M. and Devereux, J., eds.) Oxford University Press, NY (1992). Preferred methods to determine identity are designed to give the best match between the sequences tested. Methods to determine identity and similarity are codified in publicly available computer programs. Sequence alignments and percent identity calculations may be performed using the Megalign program of the LASERGENE bioinformatics computing suite (DNASTAR, Inc., Madison, Wisconsin). Multiple alignment of the sequences can also be performed using the Clustal method of alignment (Higgins and Sharp CABIOS, 5, 151-153 (1989) with default parameters (GAP PENALTY=10, GAP LENGTH PENALTY=10). Relevant programs also include the GCG suite of programs (Wisconsin Package Version 9.0, Genetics Computer Group (GCG), Madison, Wisconsin); BLASTP, BLASTN, BLASTX (Altschul, et al., J. Mol. Biol. 215:403-410 (1990); DNASTAR (DNASTAR, Inc., Madison, Wisconsin); and the FASTA program incorporating the Smith-Waterman algorithm (Pearson, Comput. Methods Genome Res., [Proc. Int. Symp.] (1994), Meeting Date 1992, 111-20. Editor(s): Suhai, Sandor. Publisher: Plenum, New York, N.Y. Within the context of this disclosure it will be understood that where sequence analysis software is used for analysis, the results of the analysis are based on the “default values” of the program referenced. As used herein “default values” will mean any set of values or parameters, which originally load with the software when first initialized.

Variants also include nucleic acid molecules that hybridizes under stringent hybridization conditions to a sequence disclosed herein and provide the same function as the reference sequence. Exemplary stringent hybridization conditions include an overnight incubation at 42° C. in a solution including 50% formamide, 5×SSC (750 mM NaCl, 75 mM trisodium citrate), 50 mM sodium phosphate (pH 7.6), 5XDenhardt's solution, 10% dextran sulfate, and 20 μg/ml denatured, sheared salmon sperm DNA, followed by washing the filters in 0.1×SSC at 50° C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37° C. in a solution including 6XSSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

“Specifically binds” refers to an association of a binding domain (of, for example, a CAR binding domain or a nanoparticle selected cell targeting ligand) to its cognate binding molecule with an affinity or Ka (i.e., an equilibrium association constant of a particular binding interaction with units of 1/M) equal to or greater than 10⁵ M⁻¹, while not significantly associating with any other molecules or components in a relevant environment sample. “Specifically binds” is also referred to as “binds” herein. Binding domains may be classified as “high affinity” or “low affinity”. In particular embodiments, “high affinity” binding domains refer to those binding domains with a Ka of at least 10⁷ M⁻¹, at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹. In particular embodiments, “low affinity” binding domains refer to those binding domains with a Ka of up to 10⁷ M−1, up to 10⁶ M−1, up to 10⁵ M−1. Alternatively, affinity may be defined as an equilibrium dissociation constant (Kd) of a particular binding interaction with units of M (e.g., 10⁻⁵ M to 10⁻¹³ M). In certain embodiments, a binding domain may have “enhanced affinity,” which refers to a selected or engineered binding domains with stronger binding to a cognate binding molecule than a wild type (or parent) binding domain. For example, enhanced affinity may be due to a Ka (equilibrium association constant) for the cognate binding molecule that is higher than the reference binding domain or due to a Kd (dissociation constant) for the cognate binding molecule that is less than that of the reference binding domain, or due to an off-rate (Koff) for the cognate binding molecule that is less than that of the reference binding domain. A variety of assays are known for detecting binding domains that specifically bind a particular cognate binding molecule as well as determining binding affinities, such as Western blot, ELISA, and BIACORE® (GE Healthcare, United States) analysis (see also, e.g., Scatchard, et al., 1949, Ann. N.Y. Acad. Sci. 51:660; and U.S. Pat. Nos. 5,283,173, 5,468,614, or the equivalent).

Unless otherwise indicated, the practice of the present disclosure can employ conventional techniques of immunology, molecular biology, microbiology, cell biology and recombinant DNA. These methods are described in the following publications. See, e.g., Sambrook, et al. Molecular Cloning: A Laboratory Manual, 2nd Edition (1989); F. M. Ausubel, et al. eds., Current Protocols in Molecular Biology, (1987); the series Methods IN Enzymology (Academic Press, Inc.); M. MacPherson, et al., PCR: A Practical Approach, IRL Press at Oxford University Press (1991); MacPherson et al., eds. PCR 2: Practical Approach, (1995); Harlow and Lane, eds. Antibodies, A Laboratory Manual, (1988); and R. I. Freshney, ed. Animal Cell Culture (1987).

As will be understood by one of ordinary skill in the art, each embodiment disclosed herein can comprise, consist essentially of or consist of its particular stated element, step, ingredient or component. Thus, the terms “include” or “including” should be interpreted to recite: “comprise, consist of, or consist essentially of.” The transition term “comprise” or “comprises” means has, but is not limited to, and allows for the inclusion of unspecified elements, steps, ingredients, or components, even in major amounts. The transitional phrase “consisting of” excludes any element, step, ingredient or component not specified. The transition phrase “consisting essentially of” limits the scope of the embodiment to the specified elements, steps, ingredients or components and to those that do not materially affect the embodiment. A material effect would cause a statistically significant reduction in ability of elicited antibodies to neutralize virus.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. When further clarity is required, the term “about” has the meaning reasonably ascribed to it by a person skilled in the art when used in conjunction with a stated numerical value or range, i.e. denoting somewhat more or somewhat less than the stated value or range, to within a range of ±20% of the stated value; ±19% of the stated value; ±18% of the stated value; ±17% of the stated value; ±16% of the stated value; ±15% of the stated value; ±14% of the stated value; ±13% of the stated value; ±12% of the stated value; ±11% of the stated value; ±10% of the stated value; ±9% of the stated value; ±8% of the stated value; ±7% of the stated value; ±6% of the stated value; ±5% of the stated value; ±4% of the stated value; ±3% of the stated value; ±2% of the stated value; or ±1% of the stated value. In particular embodiments, the residue numbering of mutation and deletion positions of Env is precise, rather than approximate.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents, printed publications, journal articles and other written text throughout this specification (referenced materials herein). Each of the referenced materials are individually incorporated herein by reference in their entirety for their referenced teaching.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

The particulars shown herein are by way of example and for purposes of illustrative discussion of the preferred embodiments of the present invention only and are presented in the cause of providing what is believed to be the most useful and readily understood description of the principles and conceptual aspects of various embodiments of the invention. In this regard, no attempt is made to show structural details of the invention in more detail than is necessary for the fundamental understanding of the invention, the description taken with the drawings and/or examples making apparent to those skilled in the art how the several forms of the invention may be embodied in practice.

Definitions and explanations used in the present disclosure are meant and intended to be controlling in any future construction unless clearly and unambiguously modified in the examples or when application of the meaning renders any construction meaningless or essentially meaningless. In cases where the construction of the term would render it meaningless or essentially meaningless, the definition should be taken from Webster's Dictionary, 3rd Edition or a dictionary known to those of ordinary skill in the art, such as the Oxford Dictionary of Biochemistry and Molecular Biology (Eds. Attwood T et al., Oxford University Press, Oxford, 2006).

What is claimed is:

1. A method of eliciting antibodies that bind full length glycosylated human immunodeficiency virus (HIV) envelope protein (Env) in a subject in need thereof, the method comprising
administering to the subject an HIV Env that binds germline (gl) B cell receptors (BCR); and
ceasing an anti-viral therapy to the subject, thereby eliciting antibodies that bind full length glycosylated HIV Env.

2. The method of claim 1, wherein the ceasing of the anti-viral therapy is at least 2 weeks after the administering.

3. The method of claim 1, wherein the ceasing of the anti-viral therapy is within 8 weeks after the administering.

4. The method of claim 1, wherein the ceasing of the anti-viral therapy is 2, 3, 4, 5, 6, 7, or 8 weeks after the administering.

5. The method of claim 1, wherein the ceasing of the anti-viral therapy is 2, 3, or 4 weeks after the administering.

6. The method of claim 1, wherein the ceasing of the anti-viral therapy is 14, 15, 16, 17, 18, 19, 20, 21, 22, 23, 24, 25, 16, 27, 28, 29, 30, 31, 32, 33, 34, 35, 36, 37, 38, 39, 40, 41, or 42 days after the administering.

7. The method of claim 1, wherein the ceasing of the anti-viral therapy is 14, 15, 16, 17, 18, 19, 20, 21, 22, 23, 24, 25, 16, 27, or 28 days after the administering.

8. The method of claim 1, wherein the ceasing of the anti-viral therapy is within 15 days before or 15 days after the administering.

9. The method of claim 1, wherein the ceasing of the anti-viral therapy is within 10 days before or 10 days after the administering.

10. The method of claim 1, wherein the ceasing of the anti-viral therapy is within 7 days before or 7 days after the administering.

11. The method of claim 1, wherein the ceasing of the anti-viral therapy is within 3 days before or 3 days after the administering.

12. The method of claim 1, wherein the ceasing of the anti-viral therapy is within 24 hours before or 24 hours after the administering.

13. The method of claim 1, wherein the HIV Env comprises the sequence as set forth in SEQ ID NO: 1 or a sequence having at least 98% sequence identity to the sequence as set forth in SEQ ID NO: 1.

14. The method of claim 1, wherein the HIV Env comprises: (i) mutations at one or more of: N460D, N463D, S278R, G471S, V65C, and S115C; (ii) removal of the V1 loop and removal of the V2 loop; (iii) replacement of the V3 loop with a flexible linker; and (iv) an N-terminal truncation;
wherein the HIV Env does not have a mutation at position 276.

15. The method of claim 1, wherein the HIV Env comprises mutations at N460D, N463D, S278R, G471S, V65C, and S115C.

16. The method of claim 14, wherein the N-terminal truncation is directly before residue 49, 48, 47, 46, 45, 44, 43, 42, 41, 40 or 39.

17. The method of claim 14, wherein the N-terminal truncation is directly before residue 44.

18. The method of claim 1, wherein the HIV Env further comprises a C-terminal truncation directly after residue 499, 498, 497, 496, 495, 494, 493, 492, 491, 490 or 389.

19. The method of claim 18, wherein the C-terminal truncation is directly after residue 494.

20. The method of claim 14, wherein the flexible linker comprises a sequence as set forth in any one of SEQ ID NOs: 2-72.

21. The method of claim 14, wherein the V3 loop comprises residues 296-331.

22. The method of claim 14, wherein removal of the V1 loop comprises removal of residues 131-152 and/or removal of the V2 loop comprises removal of residues 161-196.

23. The method of claim 14, wherein removal of the V1 loop and removal of the V2 loop comprises removal of residues 123-196.

24. The method of claim 1, wherein the HIV Env retains glycosylation at N463 but lacks glycosylation at N276 and N460.

25. The method of claim 1, wherein the HIV Env retains glycosylation at position 463.

26. The method of claim 1, wherein the HIV Env comprises a sequence as set forth in any one of SEQ ID NOs: 73-116 or a sequence having at least 98% sequence identity to a sequence as set forth in any one of SEQ ID NOs: 73-116.

27. The method of claim 1, wherein the HIV Env is multimerized.

28. The method of claim 27, wherein the HIV Env is multimerized with a ferritin multimerization domain.

29. The method of claim 28, wherein the ferritin multimerization domain comprises a sequence as set forth in any one of SEQ ID NOs: 117-120 or a sequence having at least 98% sequence identity to a sequence as set forth in any one of SEQ ID NOs: 117-120.

30. The method of claim 27, wherein the HIV Env is multimerized with a C4b multimerization domain.

31. The method of claim 30, wherein the C4b multimerization domain comprises a sequence as set forth in any one of SEQ ID NOs: 122-154 or a sequence having at least 98% sequence identity to a sequence as set forth in any one of SEQ ID NOs: 122-154.

32. The method of claim 27, wherein the HIV Env is multimerized into a 5-mer, a 6-mer, or a heptamer.

33. The method of claim 1, wherein the HIV Env is multimerized into a heptamer with a heptamerization domain.

34. The method of claim 33, wherein the heptamerization domain comprises a sequence as set forth in any one of SEQ ID NOs: 117-120 or a sequence having at least 98% sequence identity to a sequence as set forth in any one of SEQ ID NOs: 117-120.

35. The method of claim 27, wherein the HIV Env is multimerized into a 24-mer.

36. The method of claim 1, wherein the HIV Env comprises a 426c core, linker, and ferritin multimerization domain.

37. The method of claim 36, wherein the HIV Env comprises a sequence as set forth in SEQ ID NO: 121 or a sequence having at least 98% sequence identity to the sequence as set forth in SEQ ID NO: 121.

38. The method of claim 1, wherein the HIV Env comprises a 426c core, linker, and C4b multimerization domain.

39. The method of claim 38, wherein the HIV Env comprises a sequence as set forth in SEQ ID NO: 159 or SEQ ID NO: 160 or a sequence having at least 98% sequence identity to the sequence as set forth in SEQ ID NO: 159 or SEQ ID NO: 160.

40. The method of claim 1, wherein the glBCR comprise glVRC01 BCR.

41. The method of claim 1, further comprising administering a boost immunization comprising an HIV Env to the subject.

42. The method of claim 41, wherein the ceasing of the anti-viral therapy is at least 2 weeks after the administering of the boost immunization.

43. The method of claim 41, wherein the ceasing of the anti-viral therapy is within 8 weeks after the administering of the boost immunization.

44. The method of claim 41, wherein the ceasing of the anti-viral therapy is 2, 3, 4, 5, 6, 7, or 8 weeks after the administering of the boost immunization.

45. The method of claim 41, wherein the ceasing of the anti-viral therapy is 2, 3, or 4 weeks after the administering of the boost immunization.

46. The method of claim 41, wherein the ceasing of the anti-viral therapy is 14, 15, 16, 17, 18, 19, 20, 21, 22, 23, 24, 25, 16, 27, 28, 29, 30, 31, 32, 33, 34, 35, 36, 37, 38, 39, 40, 41, or 42 days after the administering of the boost immunization.

47. The method of claim 41, wherein the ceasing of the anti-viral therapy is 14, 15, 16, 17, 18, 19, 20, 21, 22, 23, 24, 25, 16, 27, or 28 days after the administering of the boost immunization.

48. The method of claim 41, wherein the ceasing of the anti-viral therapy is within 15 days before or 15 days after the administering of the boost immunization.

49. The method of claim 41, wherein the ceasing of the anti-viral therapy is within 10 days before or 10 days after the administering of the boost immunization.

50. The method of claim 41, wherein the ceasing of the anti-viral therapy is within 7 days before or 7 days after the administering of the boost immunization.

51. The method of claim 41, wherein the ceasing of the anti-viral therapy is within 3 days before or 3 days after the administering of the boost immunization.

52. The method of claim 41, wherein the ceasing of the anti-viral therapy is within 24 hours before or 24 hours after the administering of the boost immunization.

53. The method of claim 41, wherein the boost immunization comprises an HIV Env with an NLGS at position 276.

54. The method of claim 41, wherein the boost immunization comprises a same HIV Env as administered according to claim 13.

55. The method of claim 41, wherein the boost immunization comprises a different HIV Env as administered according to claim 13.

56. The method of claim 41, wherein a time between the administering of claim 1 and the administering the boost immunization of claim 35 is 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 1 month, 5 weeks, 6 weeks, 7 weeks, 2 months, 3 months, 4 months, 5 months, or 6 months.

57. The method of claim 41, wherein the time between the administering of claim 1 and the administering the boost immunization of claim 41 is 1, 2, or 3 months.

58. A method of claim 41, wherein the time between the administering of claim 1 and the administering the boost immunization of claim 41 is 1, 2, or 3 months after a re-initiation of an anti-viral therapy.

59. The method of claim 1, further comprising administering an adjuvant to the subject.

60. The method of claim 59, wherein the administering of the adjuvant is with the administering of the HIV-Env.

61. The method of claim 59, wherein the administering of the adjuvant is with a boost immunization.

62. The method of claim 59, wherein the adjuvant comprises one or more of PolyIC, Adjuplex, Alum, Ribi, or GLA-LSQ.

63. The method of claim 1, wherein the subject in need thereof received an HIV vaccination before the administering.

64. The method of claim 63, wherein the subject in need thereof received the HIV vaccination before the subject contracted HIV.

65. The method of claim 63, wherein the subject in need thereof received the HIV vaccination after the subject contracted HIV.

66. The method of claim 63, wherein the subject in need thereof received the HIV vaccination before the subject contracted HIV, and received a second HIV vaccination after the subject contracted HIV and before the administering.

67. The method of claim 1, wherein the anti-viral therapy is an anti-retroviral therapy.

68. The method of claim 1, wherein the anti-viral therapy comprises a nucleoside reverse transcriptase inhibitor, a non-nucleoside reverse transcriptase inhibitor, a protease inhibitor, a fusion inhibitor, a CCR5 antagonist, an integrase strand transfer inhibitor, an attachment inhibitor, a post-attachment inhibitor, a capsid inhibitor, a pharmacokinetic enhancer, or a combination treatment.

69. The method of claim 68,
wherein the nucleoside reverse transcriptase inhibitor comprises abacavir, emtricitabine, lamivudine, tenofovir disoproxil, fumarate, or zidovudine;
wherein the non-nucleoside reverse transcriptase inhibitor comprises doravirine, efavirenz, etravirine, nevirapine, or rilpivirine;
wherein the protease inhibitor comprises atazanavir, darunavir, fosamprenavir, ritonavir or tipranavir;
wherein the fusion inhibitor comprises enfuvirtide;
wherein the CCR5 antagonist comprises maraviroc;
wherein the integrase strand transfer inhibitor comprises cabotegravir, dolutegravir, or raltegravir; wherein the attachment inhibitor comprises fostemsavir;
wherein the post-attachment inhibitor comprises ibalizumab;
wherein the capsid inhibitor comprises lenacapavir;
wherein the pharmacokinetic enhancer comprises cobicistat; or
wherein the combination treatment comprises abacavir and lamivudine; abacavir, dolutegravir, and lamivudine; abacavir, lamivudine, and zidovudine; atazanavir and cobicistat; bictegravir, emtricitabine, and tenofovir alafenamide; cabotegravir and rilpivirine; darunavir and cobicistat; darunavir, cobicistat, emtricitabine, and tenofovir alafenamide; dolutegravir and lamivudine; dolutegravir and rilpivirine; doravirine, lamivudine, and tenofovir disoproxil fumarate; efavirenz, emtricitabine, and tenofovir disoproxil fumarate; efavirenz, lamivudine, and tenofovir disoproxil fumarate; efavirenz, lamivudine, and tenofovir disoproxil fumarate; elvitegravir, cobicistat, emtricitabine, and tenofovir alafenamide; elvitegravir, cobicistat, emtricitabine, and tenofovir disoproxil fumarate; emtricitabine, rilpivirine, and tenofovir alafenamide; emtricitabine, rilpivirine, and tenofovir disoproxil fumarate; emtricitabine and tenofovir alafenamide; emtricitabine and tenofovir disoproxil fumarate; lamivudine and tenofovir disoproxil fumarate; lamivudine and zidovudine; or lopinavir and ritonavir.


SEQUENCE LISTING (160 sequences)

SEQ ID NO: 1 - protein, 347 aa, synthetic construct
VWKEAKTTLFCASDAKAYEKECHNVWATHACVPTDPNPQEVVLENVTENFNMWKNDMVDQMQEDVISIWDQCLKPCVKLTNTSTLTQACPKVTFDPIPIHYCAPAGYAILKCNNKTFNGKGPCNNVSTVQCTHGIKPVVSTQLLLNGSLAEEEIVIRSKNLRDNAKIIIVQLNKSVEIVCTRPNNGGSGSGGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPSGGDMRDNWRSELYKYKVVEIKPL

SEQ ID NO: 2 - protein, 6 aa, synthetic construct
GGSGSG

SEQ ID NO: 3 - protein, 4 aa, synthetic construct
  MOD_RES 1: note = N-protected amino acid
CGGG

SEQ ID NO: 4 - protein, 18 aa, synthetic construct
GSTSGSGKPGSGEGSTKG

SEQ ID NO: 5 - protein, 7 aa, synthetic construct
SGRAHAG

SEQ ID NO: 6 - protein, 4 aa, synthetic construct
GGGG

SEQ ID NO: 7 - protein, 5 aa, synthetic construct
GGGGG

SEQ ID NO: 8 - protein, 6 aa, synthetic construct
GGGGGG

SEQ ID NO: 9 - protein, 7 aa, synthetic construct
GGGGGGG

SEQ ID NO: 10 - protein, 8 aa, synthetic construct
GGGGGGGG

SEQ ID NO: 11 - protein, 9 aa, synthetic construct
GGGGGGGGG

SEQ ID NO: 12 - protein, 10 aa, synthetic construct
GGGGGGGGGG

SEQ ID NO: 13 - protein, 4 aa, synthetic construct
SSSS

SEQ ID NO: 14 - protein, 5 aa, synthetic construct
SSSSS

SEQ ID NO: 15 - protein, 6 aa, synthetic construct
SSSSSS

SEQ ID NO: 16 - protein, 7 aa, synthetic construct
SSSSSSS

SEQ ID NO: 17 - protein, 8 aa, synthetic construct
SSSSSSSS

SEQ ID NO: 18 - protein, 9 aa, synthetic construct
SSSSSSSSS

SEQ ID NO: 19 - protein, 10 aa, synthetic construct
SSSSSSSSSS

SEQ ID NO: 20 - protein, 4 aa, synthetic construct
AAAA

SEQ ID NO: 21 - protein, 5 aa, synthetic construct
AAAAA

SEQ ID NO: 22 - protein, 6 aa, synthetic construct
AAAAAA

SEQ ID NO: 23 - protein, 7 aa, synthetic construct
AAAAAAA

SEQ ID NO: 24 - protein, 8 aa, synthetic construct
AAAAAAAA

SEQ ID NO: 25 - protein, 9 aa, synthetic construct
AAAAAAAAA

SEQ ID NO: 26 - protein, 10 aa, synthetic construct
AAAAAAAAAA

SEQ ID NO: 27 - protein, 4 aa, synthetic construct
GSGS

SEQ ID NO: 28 - protein, 6 aa, synthetic construct
GSGSGS

SEQ ID NO: 29 - protein, 8 aa, synthetic construct
GSGSGSGS

SEQ ID NO: 30 - protein, 10 aa, synthetic construct
GSGSGSGSGS

SEQ ID NO: 31 - protein, 12 aa, synthetic construct
GSGSGSGSGSGS

SEQ ID NO: 32 - protein, 14 aa, synthetic construct
GSGSGSGSGSGSGS

SEQ ID NO: 33 - protein, 16 aa, synthetic construct
GSGSGSGSGSGSGSGS

SEQ ID NO: 34 - protein, 18 aa, synthetic construct
GSGSGSGSGSGSGSGSGS

SEQ ID NO: 35 - protein, 20 aa, synthetic construct
GSGSGSGSGSGSGSGSGSGS

SEQ ID NO: 36 - protein, 4 aa, synthetic construct
GSSG

SEQ ID NO: 37 - protein, 8 aa, synthetic construct
GSSGGSSG

SEQ ID NO: 38 - protein, 12 aa, synthetic construct
GSSGGSSGGSSG

SEQ ID NO: 39 - protein, 16 aa, synthetic construct
GSSGGSSGGSSGGSSG

SEQ ID NO: 40 - protein, 20 aa, synthetic construct
GSSGGSSGGSSGGSSGGSSG

SEQ ID NO: 41 - protein, 24 aa, synthetic construct
GSSGGSSGGSSGGSSGGSSGGSSG

SEQ ID NO: 42 - protein, 28 aa, synthetic construct
GSSGGSSGGSSGGSSGGSSGGSSGGSSG

SEQ ID NO: 43 - protein, 32 aa, synthetic construct
GSSGGSSGGSSGGSSGGSSGGSSGGSSGGSSG

SEQ ID NO: 44 - protein, 36 aa, synthetic construct
GSSGGSSGGSSGGSSGGSSGGSSGGSSGGSSGGSSG

SEQ ID NO: 45 - protein, 40 aa, synthetic construct
GSSGGSSGGSSGGSSGGSSGGSSGGSSGGSSGGSSGGSSG

SEQ ID NO: 46 - protein, 6 aa, synthetic construct
GSGGSG

SEQ ID NO: 47 - protein, 9 aa, synthetic construct
GSGGSGGSG

SEQ ID NO: 48 - protein, 12 aa, synthetic construct
GSGGSGGSGGSG

SEQ ID NO: 49 - protein, 15 aa, synthetic construct
GSGGSGGSGGSGGSG

SEQ ID NO: 50 - protein, 18 aa, synthetic construct
GSGGSGGSGGSGGSGGSG

SEQ ID NO: 51 - protein, 21 aa, synthetic construct
GSGGSGGSGGSGGSGGSGGSG

SEQ ID NO: 52 - protein, 24 aa, synthetic construct
GSGGSGGSGGSGGSGGSGGSGGSG

SEQ ID NO: 53 - protein, 27 aa, synthetic construct
GSGGSGGSGGSGGSGGSGGSGGSGGSG

SEQ ID NO: 54 - protein, 30 aa, synthetic construct
GSGGSGGSGGSGGSGGSGGSGGSGGSGGSG

SEQ ID NO: 55 - protein, 6 aa, synthetic construct
GSSGSS

SEQ ID NO: 56 - protein, 9 aa, synthetic construct
GSSGSSGSS

SEQ ID NO: 57 - protein, 12 aa, synthetic construct
GSSGSSGSSGSS

SEQ ID NO: 58 - protein, 15 aa, synthetic construct
GSSGSSGSSGSSGSS

SEQ ID NO: 59 - protein, 18 aa, synthetic construct
GSSGSSGSSGSSGSSGSS

SEQ ID NO: 60 - protein, 21 aa, synthetic construct
GSSGSSGSSGSSGSSGSSGSS

SEQ ID NO: 61 - protein, 24 aa, synthetic construct
GSSGSSGSSGSSGSSGSSGSSGSS

SEQ ID NO: 62 - protein, 27 aa, synthetic construct
GSSGSSGSSGSSGSSGSSGSSGSSGSS

SEQ ID NO: 63 - protein, 30 aa, synthetic construct
GSSGSSGSSGSSGSSGSSGSSGSSGSSGSS

SEQ ID NO: 64 - protein, 4 aa, synthetic construct
GAGA

SEQ ID NO: 65 - protein, 6 aa, synthetic construct
GAGAGA

SEQ ID NO: 66 - protein, 8 aa, synthetic construct
GAGAGAGA

SEQ ID NO: 67 - protein, 10 aa, synthetic construct
GAGAGAGAGA

SEQ ID NO: 68 - protein, 12 aa, synthetic construct
GAGAGAGAGAGA

SEQ ID NO: 69 - protein, 14 aa, synthetic construct
GAGAGAGAGAGAGA

SEQ ID NO: 70 - protein, 16 aa, synthetic construct
GAGAGAGAGAGAGAGA

SEQ ID NO: 71 - protein, 18 aa, synthetic construct
GAGAGAGAGAGAGAGAGA

SEQ ID NO: 72 - protein, 20 aa, synthetic construct
GAGAGAGAGAGAGAGAGAGA

SEQ ID NO: 73 - protein, 183 aa, synthetic construct
RPVVSTQLLLNGSLAEEEVVIRSVNFTDNAKTIIVQLNTSVEINCTGAGHCNISRAKWNNTLKQIASKLREQFGNNKTIIFKQSSGGDPEIVTHSFNCGGEFFYCDSTQLFNSTWFNSTWSTEGSNNTEGSDTITLPCRPAPPPHCSSNITGLLLTRDGGNSNNESEIFRPGGGDMRDNWRSE

SEQ ID NO: 74 - protein, 183 aa, synthetic construct
RPVVSTQLLLNGSLAEEEVVIRSVNFTDNAKTIIVQLNTSVEINCTGAGHCNISRAKWNNTLKQIASKLREQFGNNKTIIFKQSSGGDPEIVTHSFNCGGEFFYCNSTQLFNSTWFNSTWSTEGSNNTEGSDTITLPCRPAPPPHCSSNITGLLLTRDGGNSNNESEIFRPGGGDMRDNWRSE

SEQ ID NO: 75 - protein, 175 aa, synthetic construct
DTITLPCRPAPPPHCSSNITGLLLTRDGGNSNNESEIFRPGGGDMRDNWRSGLSGPVVSTQLLLNGSLAEEEVVIRSVNFTDNAKTIIVQLNTSVEINCTGAGHCNISRAKWNNTLKQIASKLREQFGNNKTIIFKQSSGGDPEIVTHSFNCGGEFFYCDSTQLFNSTWFNSTWS

SEQ ID NO: 76 - protein, 185 aa, synthetic construct
GEFFYCDSTQLFNSTWFNSTWSTEGSNNTEGSDTITLPCRPAPPPHCSSNITGLLLTRDGGNSNNESEIFRPGGGDMRDNWRSGLSGPVVSTQLLLNGSLAEEEVVIRSVNFTDNAKTIIVQLNTSVEINCTGAGHCNISRAKWNNTLKQIASKLREQFGNNKTIIFKQSSGGDPEIVTHSFNCG

SEQ ID NO: 77 - protein, 175 aa, synthetic construct
DTITLPCRPAPPPHCSSNITGLILTRDGGNSNNESEIFRPGGGDMRDIARCQIAGTVVSTQLLLNGSLAEEEVVIRSVNFTDNAKSICVQLNTSVEINCTGAGHCNISRAKWNNTLKQIASKLREQFGNNKTIIFKQSSGGDPEIVTHSFNCGGEFFYCDSTQLFNSTWFNSTWS

SEQ ID NO: 78 - protein, 175 aa, synthetic construct
DTITLPCRPAPPPHCSSNITGLILTRDGGNSNNESEIFRPGGGDMRDIARCQIAGTVVSTQLLLNGSLAEEEVVCRSVNFTDNAKSICVQLNTSVEINCTGAGHCNISRAKWNNTLKQIASCLREQFGNNKTIIFKQSSGGDPEIVTHSFNCGGEFFYCDSTQLFNSTWFNSTWS

SEQ ID NO: 79 - protein, 168 aa, synthetic construct
DTITLPCRPAPPPHCSSNITGLILTRDGGNSNNESEIFRPGGGDMAGMPRCGGGAVSTQLLLNGSLAEEEVVCRSVNFTDNAKSICVQLNTSVEINCTGAGHCNISRAKWNNTLKQIASCLREQFGNNKTIIFKQSSGGDPEIVTHSFNCGGEFFYCDSTQLFNSTWF

SEQ ID NO: 80 - protein, 175 aa, synthetic construct
DTITLPCRPAPPPHCSSNITGLILTRDGGNSNDESEIFRPGGGDMRDIARCQIAGTVVSTQLLLNGSLAEEEVVIRSVDFTDNAKSICVQLDTSVEIDCTGAGHCDISRAKWDNTLKQIASKLREQFGNDKTIIFKQSSGGDPEIVTHSFNCGGEFFYCDSTQLFDSTWFDSTWS

SEQ ID NO: 81 - protein, 185 aa, synthetic construct
GEFFYCDSTQLFNSTWFNSTWSTEGSNNTEGSDTITLPCRPAPPPHCSSNITGLILTRDGGNSNNESEIFRPGGGDMRDIARCQIAGTVVSTQLLLNGSLAEEEVVIRSVNFTDNAKSICVQLNTSVEINCTGAGHCNISRAKWNNTLKQIASKLREQFGNNKTIIFKQSSGGDPEIVTHSFNCG

SEQ ID NO: 82 - protein, 185 aa, synthetic construct
GEFFYCDSTQLFNSTWFNSTWSTEGSNNTEGSDTITLPCRPAPPPHCSSNITGLILTRDGGNSNNESEIFRPGGGDMRCGARSGIAGTVVSTQLLLNGSLAEEEVVIRSVNFTDNAKCIIVQLNTSVEINCTGAGHCNISRAKWNNTLKQIASKLREQFGNNKTIIFKQSSGGDPEIVTHSFNCG

SEQ ID NO: 83 - protein, 185 aa, synthetic construct
GEFFYCDSTQLFNSTWFNSTWSTEGSNNTEGSDTITLPCRPAPPPHCSSNITGLILTRDGGNSNNESEIFRPGGGDMRDIARCQIAGTVVSTQLLLNGSLAEEEVVCRSVNFTDNAKSICVQLNTSVEINCTGAGHCNISRAKWNNTLKQIASCLREQFGNNKTIIFKQSSGGDPEIVTHSFNCG

SEQ ID NO: 84 - protein, 174 aa, synthetic construct
DTITLPCRPAPPPHCSSNITGLILTRDGGTSDDKTEIFRPGGGDMRDIARCQIAGTVVSTQLLLNGSLAEEEVVIRSEDFRDNAKSICVQLNTSVEINCTGAGHCNISRAKWNNTLKQIASKLREQFGNKTIIFKQSSGGDPEFVTHSFNCGGEFFYCDSTQLFDSTWFDSTWS

SEQ ID NO: 85 - protein, 174 aa, synthetic construct
DTITLPCRPAPPPHCSSNITGLILTRGGGISDDKTEIFRPGGGDMRDIARCQIAGTVVSTQLLLNGSLAEEEVVIRSEDFRDNSKSICVQLNTSVEINCTGAGHCNISRAKWNNTLKQIASKLREQFGNKTIIFSQSLGGDPEFVTHSFNCGGEFFYCDSTQLFDSTWFDSTWS

SEQ ID NO: 86 - protein, 174 aa, synthetic construct
DTITLPCRPAPPPHCSSNITGLILTRGGGISDDKTEIFRPGGGDMRDIARCQIAGTVVSTQLLLNGSLAEEEVVIRSENFTDNSKSICVQLNTSVEINCTGAGHCNISRAKWNNTLKQIASKLREQFGNKTIIFSQSLGGDPEFVTHSFNCGGEFFYCDSTQLFDSTWFDSTWS

SEQ ID NO: 87 - protein, 173 aa, synthetic construct
DTITLPCRPAPPPHCSSNITGLILTRGGGISDDKTEIFRPSGGDMRDIARCQIAGTVVSTQLLLNGSLAEEEVVIRSEDFRDNSKSICVQLNTSVEINCTGAGHCNISRAKWNNTLKQIASKLREQFGNKTIIFSQSLGGDPEFVTHSFNCGGEFFYCDSTQLFDSTWFDSTW

SEQ ID NO: 88 - protein, 173 aa, synthetic construct
DTITLPCRPAPPPHCSSNITGLILTRGGGISDDKTEIFRPGGGDMRDIARCQIAGTVVSTQLLLNGSLAEEEVVIRSEDFRDNSKSICVQLNTSVEINCTGAGHCNISRAKWNNTLKQIASKLREQFGNRTIIFSQSLGGDPEFVTHSFNCGGEFFYCDSTQLFDSTWFDSTW

SEQ ID NO: 89 - protein, 173 aa, synthetic construct
DTITLPCRPAPPPHCSSNITGLILTRGGGISDDDTEIFRPSGGDMRDIARCQIAGTVVSTQLFLNGSLAEEEVVIRSEDFRDNSKSICVQLNTSVEINCTGAGHCNISRAKWNNTLKQIASKLREQFGNRTIIFSQSLGGDPEFVTHSFNCGGEFFYCDSTQLFDSTWFDSTW

SEQ ID NO: 90 - protein, 173 aa, synthetic construct
DTITLPCRPAPPPHCSSNITGLILTRAGGISDDNTEIFRPSGGDMRDIARCQIAGTVVSTQLFLNGSLAEEEVVIRSEDFRDNSKSICVQLNTSVEINCTGAGHCNISRAKWNNTLKQIASKLREQFGNRTIIFSQSLGGDPEFVTHSFNCGGEFFYCDSTQLFDSTWFDSTW

SEQ ID NO: 91 - protein, 173 aa, synthetic construct
DTITLPCRPAPPPHCSSNITGLILTRGGGISDDNTEIFRPSGGDMRDIARCQIAGTVVSTQLLLNGSLAEEEVVIRSEDFRDNSKSICVQLNTSVEINCTGAGHCNISRAKWNNTLKQIASKLREQFGNRTIIFSQSLGGDPEFVTHSFNCGGEFFYCDSTQLFDSTWFDSTW

SEQ ID NO: 92 - protein, 173 aa, synthetic construct
DTITLPCRPAPPPHCSSNITGLILTRGGGVSDDDTEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFSQSSGGDPEFVTHSFNCGGEFFYCDSTQLFDSTWFDSTW

SEQ ID NO: 93 - protein, 173 aa, synthetic construct
DTITLPCRPAPPPHCSSNITGLILTRGGGVSDDDTEIFRPAGGDMRDIARCQIAGTVVSTQLFLNGSLAEEEVVIRSVDFRDNAKSICVQLNTSVEINCTGAGHCNISRAKWNNTLKQIASKLREQFGNRTIIFSQSSGGDPEFVTHSFNCGGEFFYCDSTQLFDSTWFDSTW

SEQ ID NO: 94 - protein, 170 aa, synthetic construct
DTITLPCRPAPPPHCSSNITGLILTRGGGVSDDDTEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFSQSSGGDPEFVTHSFNCGGEFFYCDSTQLFDSTWFD

SEQ ID NO: 95 - protein, 172 aa, synthetic construct
DTITLPCRPAPPPHCSSNITGLILTRDGGVSNDETEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCDSTQLFNSTWFNST

SEQ ID NO: 96 - protein, 172 aa, synthetic construct
DTITLPCRPAPPPHCSSNITGLILTRDGGNSNDETEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCDSTQLFNSTWFNST

SEQ ID NO: 97 - protein, 172 aa, synthetic construct
DTITLPCRPAPPPHCSSNITGLILTRDGGVSNDESEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCDSTQLFNSTWFNST

SEQ ID NO: 98 - protein, 172 aa, synthetic construct
DTITLPCRPAPPPHCSSNITGLILTRDGGVSNDETEIFRPGGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCDSTQLFNSTWFNST

SEQ ID NO: 99 - protein, 172 aa, synthetic construct
DTITLPCRPAPPPHCSSNITGLILTRDGGVSNDETEIFRPSGGDMRDIARCQIAGTVVSTQLLLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCDSTQLFNSTWFNST

SEQ ID NO: 100 - protein, 172 aa, synthetic construct
DTITLPCRPAPPPHCSSNITGLILTRDGGVSNDETEIFRPSGGDMRDIARCQIAGTVVSTQLFLNGSLAEEEVVIRSVDFTDNAKSICVQLNTSVEINCTGAGHCNISRAKWNNTLKQIASKLREQFGNRTIIFKQSSGGDPEFVTHSFNCGGEFFYCDSTQLFNSTWFNST

SEQ ID NO: 101 - protein, 172 aa, synthetic construct
DTITLPCRPAPPPHCSSNITGLILTRDGGVSNDETEIFRPSGGDMRDIARCQIAGTVVSTQLFLNGSLAEEEVVIRSVDFRDNAKSICVQLNTSVEINCTGAGHCNISRAKWNNTLKQIASKLREQFGNKTIIFKQSSGGDPEFVTHSFNCGGEFFYCDSTQLFNSTWFNST

SEQ ID NO: 102 - protein, 172 aa, synthetic construct
DTITLPCRPAPPPHCSSNITGLILTRDGGVSNDETEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEIVTHSFNCGGEFFYCDSTQLFNSTWFNST

SEQ ID NO: 103 - protein, 172 aa, synthetic construct
DTITLPCRPAPPPHCSSNITGLILTRDGGVSNDETEIFRPSGGDMRDIARCQIAGTVVSTQLFLNGSLAEEEVVIRSVDFRDNAKSICVQLDTSVEIDCTGAGHCDISRAKWDNTLKQIASKLREQFGDRTIIFKQSSGGDPEFVTHSFNCGGEFFYCDSTQLFDSTWFDST

SEQ ID NO: 104 - protein, 172 aa, synthetic construct
DTITLPCRPAPPPHCSSNITGLILTRDGGVSNNETEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCNSTQLFNSTWFNST

SEQ ID NO: 105 - protein, 172 aa, synthetic construct
DTITLPCRPAPPPHCSSNITGLILTRDGGNSNNETEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCNSTQLFNSTWFNST

SEQ ID NO: 106 - protein, 172 aa, synthetic construct
DTITLPCRPAPPPHCSSNITGLILTRDGGVSNNETEIFRPGGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCNSTQLFNSTWFNST

SEQ ID NO: 107 - protein, 172 aa, synthetic construct
DTITLPCRPAPPPHCSSNITGLILTRDGGVSNNETEIFRPSGGDMRDIARCQIAGTVVSTQLLLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCNSTQLFNSTWFNST

SEQ ID NO: 108 - protein, 172 aa, synthetic construct
DTITLPCRPAPPPHCSSNITGLILTRDGGVSNNETEIFRPSGGDMRDIARCQIAGTVVSTQLFLNGSLAEEEVVIRSVDFTDNAKSICVQLNTSVEINCTGAGHCNISRAKWNNTLKQIASKLREQFGNRTIIFKQSSGGDPEFVTHSFNCGGEFFYCNSTQLFNSTWFNST

SEQ ID NO: 109 - protein, 172 aa, synthetic construct
DTITLPCRPAPPPHCSSNITGLILTRDGGVSNNETEIFRPSGGDMRDIARCQIAGTVVSTQLFLNGSLAEEEVVIRSVDFRDNAKSICVQLNTSVEINCTGAGHCNISRAKWNNTLKQIASKLREQFGNKTIIFKQSSGGDPEFVTHSFNCGGEFFYCNSTQLFNSTWFNST

SEQ ID NO: 110 - protein, 172 aa, synthetic construct
DTITLPCRPAPPPHCSSNITGLILTRDGGVSNNETEIFRPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEIVTHSFNCGGEFFYCNSTQLFNSTWFNST

SEQ ID NO: 111 - protein, 172 aa, synthetic construct
DTITLPCRPAPPPHCSSNITGLILTRDGGVSNNETEIFRPSGGDMRDIARCQIAGTVVSTQLFLNGSLAEEEVVIRSVNFTDNAKSICVQLNTSVEINCTGAGHCNISRAKWNNTLKQIASKLREQFGNRTIIFKQSSGGDPEFVTHSFNCGGEFFYCNSTQLFNSTWFNST

SEQ ID NO: 112 - protein, 172 aa, synthetic construct
DTITLPCRPAPPPHCSSNITGLILTRAGGVSDNNTEIFFPSGGDMRDIARCQIAGTVVSTQLFLNGSLAEEEVVIRSVDFRDNAKSICVQLNTSVEINCTGAGHCNISRAKWNNTLKQIASKLREQFGNRTIIFSQSTGGDPEFVTHSFNCGGEFFYCNSTQLFNSTWFNST

SEQ ID NO: 113 - protein, 173 aa, synthetic construct
DTITLPCRPAPPPHCSSNITGLILGRAGGASDDNTEIFYPSGGDMRDIARCQIAGTVVSTQLFLNGSLAEEEVVIRSVDFRDNAKSICVQLNTSVEINCTGAGHCNISRAKWNNTLKQIASKLREQFGNRTIIFSQSTGGDPEIVTHSFNCGGEFFYCNSTQLFNSTWFNSTW

SEQ ID NO: 114 - protein, 172 aa, synthetic construct
DTITLPCRPAPPPHCSSNITGLILTRAGGVSNNETEIFFPSGGDMRDIARCQIAGTVVSTQLFLNGSLAEEEVVIRSVDFRDNAKSICVQLNTSVEINCTGAGHCNISRAKWNNTLKQIASKLREQFGNRTIIFKQSSGGDPEFVTHSFNCGGEFFYCNSTQLFNSTWFNST

SEQ ID NO: 115 - protein, 355 aa, synthetic construct
VWKEATTTLFCASDAKAYDTEVHNVWATHACVPTDPNPQEVELENVTENFNMWKNNMVEQMHEDIISLWDQSLKPCVKLTGGSVITQACPKISFEPIPIHYCAPAGFAILKCKDKKFNGKGPCSNVSTVQCTHGIRPVVSTQLLLNGSLAEEEVVIRSENFADNAKTIIVQLNESVEINCTRPNNGGSGSGGDIRQAHCNLSRAKWNDTLNKIVIKLREQFGNKTIVFKHSSGGDPEIVTHSFNCGGEFFYCNSTQLFNSTWNVTEESNNTVENNTITLPCRIKQIINMWQKVGRAMYAPPIRGQIRCSSNITGLLLTRDGGPEDNKTEVFRPGGGDMRDNWRSELYKYKVVKIE

SEQ ID NO: 116 - protein, 299 aa, synthetic construct
VWKDATTTLFCASDAKAHETEVHNVWATHACVPTDPNPQEVELKNVTENFNMWKNNMVEQMHEDIISLWDQSLKPCVKLTGGSVITQACPKVSFEPIPIHYCAPAGFAILKCKDKKFNGKGPCSNVSTVQCTHGIRPVVSTQLLLNGSLAEEEVVIRSEDFRNNAKIIIVQLNESVEINCTGAGHCNLSRAKWNDTLNKIVIKLREQFGNKTIVFKHSSGGDPEIVTHSFNCGGEFFYCNSTQLFNSTWNVTEESNNTVENNTITLPCRIKQIINMWQEVGRAMYAPPIRGQIRCSSNI

SEQ ID NO: 117 - protein, 175 aa, synthetic construct
ESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 118 - protein, 163 aa, synthetic construct
DIIKLLNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 119 - protein, 158 aa, synthetic construct
LNEQVNKEMNSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 120 - protein, 163 aa, synthetic construct
DIIKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKS

SEQ ID NO: 121 - protein, 528 aa, synthetic construct
VWKEAKTTLFCASDAKAYEKECHNVWATHACVPTDPNPQEVVLENVTENFNMWKNDMVDQMQEDVISIWDQCLKPCVKLTNTSTLTQACPKVTFDPIPIHYCAPAGYAILKCNNKTFNGKGPCNNVSTVQCTHGIKPVVSTQLLLNGSLAEEEIVIRSKNLRDNAKIIIVQLNKSVEIVCTRPNNGGSGSGGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPSGGDMRDNWRSELYKYKVVEIKPLGSGGSGESQVRQQFSKDIEKLLNEQVNKEMQSSNLYMSMSSWCYTHSLDGAGLFLFDHAAEEYEHAKKLIIFLNENNVPVQLTSISAPEHKFEGLTQIFQKAYEHEQHISESINNIVDHAIKSKDHATFNFLQWYVAEQHEEEVLFKDILDKIELIGNENHGLYLADQYVKGIAKSRKSGS

SEQ ID NO: 122 - protein, 68 aa, synthetic construct
SGRAHAGWETPEGCEQVLTGKRLMQCLPNPEDVKMALEVYKLSLEIEQLELQRDSARQSTLDKELVPR

SEQ ID NO: 123 - protein, 55 aa, synthetic construct
KKQGDADVCGEVAYIQSVVSDCHVPTAELRTLLEIRKLFLEIQKLKVELQGLSKE

SEQ ID NO: 124 - protein, 57 aa, synthetic construct
ETPEGCEQVLTGKRLMQCLPNPEDVKMALEVYKLSLEIEQLELQRDSARQSTLDKEL

SEQ ID NO: 125 - protein, 58 aa, synthetic construct
WETPEGCEQVLTGKRLMQCLPNPEDVKMALEVYKLSLEIEQLELQRDSARQSTLDKEL

SEQ ID NO: 126 - protein, 52 aa, synthetic construct
CEQVLTGKRLMQCLPNPEDVKMALEVYKLSLEIEQLELQRDSARQSTLDKEL

SEQ ID NO: 127 - protein, 57 aa, synthetic construct
ETPEGCEQVLTGKRLMQCLPNPEDVKMALEVYKLSLEIEQLELQRDSARQYTLDKEL

SEQ ID NO: 128 - protein, 57 aa, synthetic construct
ETPEGCEQVLAGKRLMQCLPNPEDVKMALEVYKLSLEIEQLELQRDRARQSTLDKEL

SEQ ID NO: 129 - protein, 57 aa, synthetic construct
ETPEGCEQVLAGKRLMQCLPNPEDVKMALEVYKLSLEIEQLELQRDRARQSTWDKEL

SEQ ID NO: 130 - protein, 57 aa, synthetic construct
EVPEGCEQVQAGRRLMQCLADPYEVKMALEVYKLSLEIELLELQRDKARKSSVLRQL

SEQ ID NO: 131 - protein, 57 aa, synthetic construct
VVPEGCEHILKGRKTMQCLPNPEDVKMALEIYKLSLDIELLELQRDRAKESTVQSPV

SEQ ID NO: 132 - protein, 55 aa, synthetic construct
EVPKDCEHVFAGKKLMQCLPNSNDVKMALEVYKLTLEIKQLQLQIDKAKHVDREL

SEQ ID NO: 133 - protein, 57 aa, synthetic construct
EYPEDCEQVHEGKKLMQCLPNLEEIKLALELYKLSLETKLLELQIDKEKKAKAKYSI

SEQ ID NO: 134 - protein, 57 aa, synthetic construct
EYPEDCEQVHEGKKLMECLPTLEEIKLALALYKLSLETNLLELQIDKEKKAKAKYST

SEQ ID NO: 135 - protein, 57 aa, synthetic construct
EIAEGCEQVLAGRKIMQCLPKPEDVRTALELYKLSLEIKQLEKKLEKEEKCTPEVQE

SEQ ID NO: 136 - protein, 67 aa, synthetic construct
EYPEGCEQVVTGRKLLQCLSRPEEVKLALEVYKLSLEIEILQTNKLKKEAFLLREREKNVTCDFNPE

SEQ ID NO: 137 - protein, 52 aa, synthetic construct
EYPEGCEQVVTGRKLLKCLSRPEEVKLALEVYKLSLEIALLELQIDKPKDAS

SEQ ID NO: 138 - protein, 52 aa, synthetic construct
EVPENCEQVIVGKKLMKCLSNPDEAQMALQLYKLSLEAELLRLQIVKARQGS

SEQ ID NO: 139 - protein, 54 aa, synthetic construct
EASEDLKPALTGNKTMQYVPNSHDVKMALEIYKLTLEVELLQLQIQKEKHTEAH

SEQ ID NO: 140 - protein, 51 aa, synthetic construct
VSAEVCEAVFKGQKLLKCLPNAMEVKMALEVYKLSLEIEKLEQEKRKLEIA

SEQ ID NO: 141 - protein, 66 aa, synthetic construct
EVPEECKQVAAGRKLLECLPNPSDVKMALEVYKLSLEIEQLEKEKYVKIQEKFSKKEMKQLTSALH

SEQ ID NO: 142 - protein, 59 aa, synthetic construct
EVLEDCRIVSRGAQLLHCLSSPEDVHRALKVYKLFLEIERLEHQKEKWIQLHRKPQSMK

SEQ ID NO: 143 - protein, 63 aa, synthetic construct
EGPEDCEIVNKGRQLLQCLSSPEDVQRALEVYKLSLEIERLEQQREKRTSVHRKAHYTKVDGP

SEQ ID NO: 144 - protein, 67 aa, synthetic construct
EAPEGCEQVLTGRKLMQCLPSPEDVKVALEVYKLSLEIKQLEKERDKLMNTHQKFSEKEEMKDLFFP

SEQ ID NO: 145 - protein, 52 aa, synthetic construct
EVPEGCEQVLTGKKLMQCLPNPEDVKMALEVYKLSLEIELLELQIDKARQGS

SEQ ID NO: 146 - protein, 47 aa, synthetic construct
GCEQVLTGKRLMQCLPNPEDVKMALEVYKLSLEIEQLELQRDSARQS

SEQ ID NO: 147 - protein, 51 aa, synthetic construct
ETPEGCEQVLTGKRLMQCLPNPEDVKMALEVYKLSLEIEQLELQRDSARQS

SEQ ID NO: 148 - protein, 53 aa, synthetic construct
GSEQVLTGKRLMQSLPNPEDVKMALEVYKLSLEIEQLELQRDSARQSTLDKEL

SEQ ID NO: 149 - protein, 57 aa, synthetic construct
ETPEGCEQVLTGKRLMQCLPNPEDVKMALEIYKLTLEIEQLELQRDSARQSTLDKEL

SEQ ID NO: 150 - protein, 57 aa, synthetic construct
ETPEGCEQVLTGKRLMQCLPNPEDVKMALEIYKLSLEIKQLELQRDSARQSTLDKEL

SEQ ID NO: 151 - protein, 50 aa, synthetic construct
EGCEQILTGKRLMQCLPDPEDVKMALEIYKLSLEIKQLELQRDRARQSTL

SEQ ID NO: 152 - protein, 57 aa, synthetic construct
ETPEGCEQVLTGKRLMQCLPNPEDVKMALEVYKLSLEIKQLELQRDRARQSTLDKEL

SEQ ID NO: 153 - protein, 52 aa, synthetic construct
EGCEQILTGKRLMQCLPNPEDVKMALEIYKLSLEIEQLELQRDRARQSTLDK

SEQ ID NO: 154 - protein, 61 aa, synthetic construct
WETPEGCEQVLTGKRLMQCLPNPEDVKMALEVYKLSLEIEQLELQRDSARQSTLDKELVPR

SEQ ID NO: 155 - protein, 4 aa, synthetic construct
AELR

SEQ ID NO: 156 - protein, 62 aa, Homo sapiens
AHAGWETPEGCEQVLTGKRLMQCLPNPEDVKMALEVYKLSLEIEQLELQRDSARQSTLDKEL

SEQ ID NO: 157 - protein, 56 aa, Gallus gallus
SKKQGDADVCGEVAYIQSVVSDCHVPTEDVKTLLEIRKLFLEIQKLKVELQGLSKE

SEQ ID NO: 158 - protein, 56 aa, synthetic construct
SKKQGDADVCGEVAYIQSVVSDCHVPTAELRTLLEIRKLFLEIQKLKVELQGLSKE

SEQ ID NO: 159 - protein, 417 aa, synthetic construct
VWKEAKTTLFCASDAKAYEKECHNVWATHACVPTDPNPQEVVLENVTENFNMWKNDMVDQMQEDVISIWDQCLKPCVKLTNTSTLTQACPKVTFDPIPIHYCAPAGYAILKCNNKTFNGKGPCNNVSTVQCTHGIKPVVSTQLLLNGSLAEEEIVIRSKNLRDNAKIIIVQLNKSVEIVCTRPNNGGSGSGGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPSGGDMRDNWRSELYKYKVVEIKPLSGSGRAHAGWETPEGCEQVLTGKRLMQCLPNPEDVKMALEVYKLSLEIEQLELQRDSARQSTLDKELVPR

SEQ ID NO: 160 - protein, 405 aa, synthetic construct
VWKEAKTTLFCASDAKAYEKECHNVWATHACVPTDPNPQEVVLENVTENFNMWKNDMVDQMQEDVISIWDQCLKPCVKLTNTSTLTQACPKVTFDPIPIHYCAPAGYAILKCNNKTFNGKGPCNNVSTVQCTHGIKPVVSTQLLLNGSLAEEEIVIRSKNLRDNAKIIIVQLNKSVEIVCTRPNNGGSGSGGDIRQAYCNISGRNWSEAVNQVKKKLKEHFPHKNISFQSSSGGDLEITTHSFNCGGEFFYCNTSGLFNDTISNATIMLPCRIKQIINMWQEVGKAIYAPPIKGNITCKSDITGLLLLRDGGDTTDNTEIFRPSGGDMRDNWRSELYKYKVVEIKPLGSSKKQGDADVCGEVAYIQSVVSDCHVPTAELRTLLEIRKLFLEIQKLKVELQGLSKE